(19) United States
(12) Patent Application Publication
(10) Pub. No.: US 2004/0180369 A1
(43) Pub. Date: Sep. 16, 2004

(54) Photothermal detection of nucleic acid hybridization

(75) Inventors: Stefan Franzen, Apex, NC (US); Daniel Feldheim, Cary, NC (US)

Correspondence Address: JENKINS & WILSON, PA, 3100 TOWER BLVD, SUITE 1400, DURHAM, NC, 27707, US

(73) Assignee: North Carolina State University

(21) Appl. No.: 10/759,496

(22) Filed: Jan. 16, 2004

Related U.S. Application Data
(60) Provisional application No. 60/515,907, filed on Oct. 30, 2003.
(60) Provisional application No. 60/440,422, filed on Jan. 16, 2003.

(51) Int. Cl. C12Q 1/68

(52) U.S. Cl. 435/6

(57) ABSTRACT

A nucleic acid hybridization detection assay is carried out at a solid surface. Capture probes comprising single-stranded oligonucleotides are immobilized to a solid substrate surface. In some embodiments using sandwich assay methodology, the capture probes hybridize complementary target nucleic acid sequences, which in turn are bound to detection probes comprising nanoparticle-oligonucleotide conjugates comprising target-complementary oligonucleotides. In some embodiments, detection probes comprise nanoparticles attached to molecules comprising one partner of a ligand-binding pair (e.g., streptavidin), while target sequences comprise the other partner of the ligand-binding pair (e.g., biotin). The solid surface is exposed to light at a wavelength that is absorbed by the nanoparticle, thus eliciting a temperature jump. The heat generated by the nanoparticle excitation is detected by a photothermography method such as infrared thermography.

### CROSS REFERENCE TO RELATED APPLICATIONS

The present patent application is related to and claims priority to U.S. Provisional Application Serial No. 60/515,907 for PHOTOTHERMAL DETECTION OF NUCLEIC ACID HYBRIDIZATION, filed Oct. 30, 2003, and to U.S. Provisional Application Serial No. 60/440,422, entitled “DETECTION OF DNA AND RNA HYBRIDIZATION BY INFRARED THERMOGRAPHY”, filed Jan. 16, 2003, which applications are incorporated herein by reference in their entirety.

### TECHNICAL FIELD

The presently described methods relate to photothermal detection of biological molecules such as nucleic acids. In particular, the methods described herein relate to the detection of nucleic acid hybridization using infrared thermography techniques combined with nanoparticle labels that provide optical contrast to permit detection.

### BACKGROUND

The detection of specific nucleic acid sequences in biological samples provides a basis for myriad practical and research techniques, including gene identification, mutation detection, gene expression profiling, and DNA sequencing. Diagnostic and forensic applications are but two areas in which nucleic acid detection techniques find widespread use.

Particular nucleic acid sequences are usually detected by one or more nucleic acid hybridization assays, in which the presence of a target sequence in a biological sample is determined by hybridizing a probe sequence designed to specifically bind the target with heterogeneous nucleic acids in the sample. The presence of the target is usually indicated by the detection of a chemical, enzymatic, magnetic or spectroscopic label that is directly or indirectly attached to either the probe or the target sequence. Such hybridization assays are increasingly being combined with parallel, high-throughput microarray technology, in which thousands of hybridization assays are carried out simultaneously on a solid substrate (e.g., a “chip”). Microarray technologies are highly amenable to automation and facilitate the screening of, for example, one biological sample against a large number of probes in a brief time period.

A broad spectrum of labeling and detection methodologies are currently used in conjunction with nucleic acid hybridization and microarray techniques. When labeled probes are used, for example, the presence of a target sequence in a biological sample is usually determined by separating hybridized and non-hybridized probe, and then directly or indirectly measuring the amount of labeled probe that is hybridized to the target. Suitable labels may provide signals detectable by luminescence, radioactivity, colorimetry, x-ray diffraction or absorption, magnetism or enzymatic activity, and can include, for example, fluorophores, chromophores, radioactive isotopes, light-scattering particles, magnetic particles, enzymes, and ligands having specific binding partners. The specific labeling method chosen depends on a multitude of factors, such as ease of attachment of the label, its sensitivity and stability over time, speed and ease of detection and quantification, and cost and safety factors.

Despite the abundance of labeling techniques, the utility, versatility and diagnostic value of any particular system for detecting nucleic acid sequences of interest can be limited. For example, fluorescent labeling and detection methodologies are generally not sufficiently sensitive to single-base mismatches in surface-bound hybridization duplexes. Additionally, fluorescence-based techniques require extensive sample preparation, as well as the use of unwieldy apparatus such as confocal microscopes. Moreover, many commonly used labeling and detection techniques have undesirably low limits of detection, thus necessitating the use of costly and time-consuming nucleic acid amplification techniques. Sensitive methods that are able to differentially detect very low concentrations of target nucleic acids thus remain in demand.

### SUMMARY

Described herein are sensitive, photothermography methods for detecting nucleic-acid sequences and nucleic acid hybridization events.

In some embodiments, provided are methods of detecting a target nucleic acid sequence, comprising: providing a hybridization complex comprising (a) a capture probe that is attached to a solid surface and (b) a target nucleic acid sequence that is hybridized to the capture probe, wherein the target nucleic acid sequence additionally comprises at least one nanoparticle attached to the target nucleic acid sequence; exposing the solid surface to light at a wavelength absorbed by the nanoparticle; and detecting a temperature of the solid surface, whereby detection of an increased temperature relative to a temperature of the solid surface that would be detected in the absence of said complex indicates the presence or amount of target nucleic acid sequence hybridized to the solid surface. Thus, in some embodiments, the use of a detection probe is not necessary.

In some embodiments, a target nucleic acid sequence hybridizes a capture oligonucleotide probe that is attached to the surface of a solid substrate. The target sequence is then hybridized with a detection probe comprising a nanoparticle, thus forming a capture probe-target sequence-detection probe hybridization complex. The hybridization complex is exposed to light (e.g., as generated by a laser) at a wavelength that is absorbed by the nanoparticle, which causes the nanoparticle to generate heat. In some embodiments, the light wavelength matches the surface plasmon resonance of the nanoparticle. The light excitation of the nanoparticle elicits a temperature jump in the environment surrounding the nanoparticle, which temperature jump (i.e., heat) is detected by photothermal techniques such as infrared thermography. The detected temperature jump provides a measure of nucleic acid hybridization at the surface, which can be correlated with the concentration of target nucleic acid present in the sample.

In some embodiments utilizing sandwich assay methodology, target sequences and capture probes comprise single-stranded nucleic acid regions, while detection probes comprise a nanoparticle-oligonucleotide conjugate. The sensitivity of the sandwich assay methodology advantageously affords the detection of nucleic acid targets that have not been amplified prior to detection.

In some embodiments, the detection probe comprises a nanoparticle attached to at least one partner of a ligand-binding pair (for example, streptavidin), while the target nucleic acid comprises the other, corresponding binding partner of the ligand-binding pair (for example, biotin). In some embodiments, the target sequence is tagged with biotin moieties during an amplification reaction in which single-stranded nucleic acid (e.g., mRNA) is used as a template, and biotin-tagged nucleotides are enzymatically incorporated into a complementary cDNA strand (e.g., by reverse transcriptase).

It is therefore an object of the present invention to provide a method of detecting nucleic acid hybridization. This object is achieved in whole or in part by the methods described in more detail below.

### BRIEF DESCRIPTION OF THE DRAWINGS

FIG. 1 is a schematic diagram illustrating a photothermal detection strategy according the methods described herein. FIG. 1 illustrates an solid surface (here, an indium tin oxide electrode, which is an illustrative, non-limiting example of a solid surface) to which a plurality of capture probes is attached. Target nucleic acid sequence (illustrated as a dotted line) is hybridized to a capture probe, and a detection probe comprising a nanoparticle and an oligonucleotide is hybridized to the target. A hybridization complex comprising a capture probe, target sequence and detection probe is thus illustrated. The hybridization complex is exposed to light energy at a wavelength that is absorbed by the nanoparticle. This irradiation elicits heat (i.e., a temperature jump) in the immediate environment of the nanoparticle, which can be detected by a thermocouple attached to the back of the solid surface (as shown), or can be detected by a charge coupled device (also shown).

FIG. 2 is an absorbance spectrum of a gold nanoparticle as a function of increasing light wavelength. When the gold nanoparticle is irradiated at 532 nm (near the calculated surface plasmon resonance of gold), a jump in absorbance is observed.

FIG. 3 is a schematic diagram of illustrating a method by which nucleic acid molecules can be attached to a nanoparticle.

FIG. 4 illustrates the formation of an amide bond by the activation of the carboxylic acid on a monolayer of 12-phosphonododecanoic acid on ITO by EDC with 5′ modified C₃NH₂ ssDNA. ITO is an illustrative, non-limiting example of a solid surface.

FIG. 5 is an x-ray photoelectron spectra (XPS) of In 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

FIG. 6 is an XPS spectra of Sn 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

FIG. 7 is an XPS N 1s spectra of ITO modified with a monolayer of 12-phosphonododecanoic acid (long dash) and ITO modified with ssDNA coupled through a monolayer of 12-phosphonododecanoic acid (short dash) fitted to a Gaussian line shape (solid).

FIG. 8 is an XPS Au 4f_{7/2,5/2} spectra of ITO modified with ssDNA coupled through a monolayer of 12-phosphonododecanoic acid (dotted line) exposed to the complementary (short dash) or non-complementary (long dash) ssDNA labeled with a 10 nm gold nanoparticle (1 nM) fitted to two Gaussian line shapes (solid).

FIG. 9 is a grazing angle reflectance FTIR spectra of ITO modified with a monolayer of 12-phosphonododecanoic acid (solid) coupled to ssDNA (dashed) recorded at an incident angle of 80 degrees with p-polarized radiation.

FIG. 10 illustrates the laser-induced temperature jump effect as manifested on a gold-nanoparticle-coated ITO electrode. FIG. 10 is a graph showing an increase in electrode temperature as a function of time, when the electrode is an ITO electrode coated with gold nanoparticles attached to the electrode surface with oligonucleotides, and when the nanoparticles are irradiated with a YAG laser at 532 nm.

FIG. 11 is a series of three infrared thermograms (8 μm-12 μm) of gold nanoparticle-coated glass slides under irradiation with 532 nm light (16 W/cm²). Particle densities were 1×10¹⁰ cm⁻², 2×¹⁰ cm⁻², and 3.5×10¹⁰ cm⁻² for A, B, and C, with recorded temperatures of 30.5° C., 35.3° C., and 42.9° C., respectively. Light-off temperature was 24.6° C. (ΔT for bare glass was <2° C.)

FIG. 12A is a schematic drawing illustrating a side view of gold nanoparticles attached to the surface of a glass slide using an aminosilane linkage.

FIG. 12B is a schematic drawing of an overhead view of the glass slide of FIG. 12A, in which gold nanoparticles have been attached in a graded density pattern to a glass slide. Particle density is greatest at the right-most end of the slight, becoming decreasingly dense towards the left-most end.

FIG. 12C is a digital photograph of the glass slide illustrated schematically in FIGS. 12A and 12B. As indicated, the slide is 64 mm in length, and density of the attached nanoparticles is greatest at the “0 mm” end, and least dense at the “64 mm” end.

FIG. 13 is a graph of light absorbance as a function of light wavelength for the attached gold nanoparticles described in FIG. 12C. That is, nanoparticles towards the end of the slide with the greatest density (“0 mm”) exhibited the overall higher absorbance, while nanoparticles towards the end of the slide with the least density (“60 mm”) exhibited the lowest, with the sample containing no particles having the lowest absorbance, as expected. All samples exhibit a marked and detectable absorbance peak at around 532 nm, where the light wavelength matches the surface plasmon resonance of the gold nanoparticles.

FIG. 14 is a graph of gold nanoparticle density (square data points) and temperature detected after irradiation by a laser at 532 nm (triangular data points), as a function of physical slide location as described in FIG. 12C. Consistent with the results shown in FIG. 13, nanoparticles towards the end of the slide with the greatest density (“0 mm”) were present in the greatest density and exhibited the highest detected temperatures, while nanoparticles towards the “64 mm” end of the slide were present in the lowest particle density and exhibited the lowest temperatures. The temperature jump (Δ T) measured for nanoparticle sample comprising 3.1×10⁵ nanoparticles was 2.5° C.

FIG. 15 is an infrared thermogram of the slide described in FIGS. 12, 13, and 14. Taken together, these four figures indicate that the amount of heat generated by gold nanoparticles attached to a solid surface and irradiated at a wavelength equal to their surface plasmon resonance is directly and predictably proportional to the concentration of nanoparticles attached at the surface.

FIG. 16 is an illustration of the limits of detection for single stranded DNA conjugated to gold nanoparticles for infrared thermography detection as described herein. In FIG. 16, temperature is graphed as a function of ssDNA-nanoparticle conjugate concentration in pM, while the background temperature of the solid substrate is indicated as a broken line. These results indicate that the limit of detection for this method is on the order of 10 fM.

FIGS. 17A and 17B, taken together, provide a graphical comparison of a known method of incorporating a fluorescent label into a target nucleic acid (FIG. 17A), and a presently-described method of incorporating one partner of ligand-binding pair (e.g., biotin) into a target nucleic acid, which ligand-binding pair partner can then be used to bind a nanoparticle to which is attached the other member of the ligand-binding pair (e.g., streptavidin) (FIG. 17B).

### DETAILED DESCRIPTION

The presently disclosed subject matter will now be described more fully hereinafter with reference to the accompanying Examples and Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which the presently disclosed subject matter belongs. All publications, patent applications, patents, and other references mentioned herein are incorporated by reference in their entirety. Throughout the specification and claims, a given chemical formula or name shall encompass all optical and stereoisomers as well as racemic mixtures where such isomers and mixtures exist.

I. General Considerations

Surface plasmon resonance (SPR) is a quantum optical-electrical phenomenon arising from the interaction of light with a metal surface. Under certain conditions, the energy carried by photons of light forces an oscillatory motion of packets of electrons on a metal's surface. Such an oscillation is known as a plasmon. The interaction of light with the conduction electrons leads to a strong absorption at the plasmon frequency. Absorption occurs only at a specific resonance wavelength of light, where the quantum energy carried by the photons exactly equals the quantum energy level of the plasmons. Often the phenomenon of plasmon resonance is treated classically as a forced harmonic oscillator problem in which the electrons are represented by a spring that has a restoring force due to the interaction with the metal nuclei.

The resonance wavelength can be determined very precisely by measuring the light reflected by a metal surface. At most wavelengths, the metal acts as a mirror, reflecting virtually all the incident light. At the wavelength that fulfills the resonance conditions, the incident light is almost completely absorbed. The wavelength at which maximum light absorption occurs is the resonance wavelength.

The coupling of light into a metal surface results in the creation of a plasmon, a group of excited electrons which behave like a single electrical entity. The plasmon, in turn, generates an electrical field which extends about 100 nanometers above the metal surface. One characteristic of SPR that makes it a useful analytical tool is that any change in the chemical composition of the environment within the range of the plasmon field causes a change in the wavelength of light that resonates with the plasmon. Stated otherwise, a change in the index of refraction near the surface results in a shift in the wavelength of light which is absorbed rather than reflected. The magnitude of the shift is quantitatively related to the magnitude of the change in the index of refraction.

Nanometer-sized metal particles such as gold and silver have intense plasmon resonances in the visible region of the electromagnetic spectrum. Excitation of gold nanoparticle plasmons causes rapid local temperature changes, which have been used previously to induce polymer gel swelling transitions (see, e.g., C. D. Jones and L. A. Lyon, J. Am. Chem. Soc. (2003) 125, 460, and S. R. Sershen et al., J. Biomed. Maer. Res. (2000) 51, 293), and to study the dynamics of melt transitions in nanometer-sized metals (see, e.g., S. Link et al., J. Phys. Chem. B (2000) 104, 6152).

II. Photothermal Detection Of Nucleic Acid Hybridization

A. General Overview Of Methods

The methods described herein are useful for detecting nucleic acid hybridization events. More particularly, the present methods can be used for detecting specific target nucleic acid sequences in a heterogeneous sample.

In some embodiments, provided are methods of detecting a target nucleic acid sequence, comprising: providing a hybridization complex comprising (a) a capture probe that is attached to a solid surface and (b) a target nucleic acid sequence that is hybridized to the capture probe, wherein the target nucleic acid sequence additionally comprises at least one nanoparticle attached to the target nucleic acid sequence; exposing the solid surface to light at a wavelength absorbed by the nanoparticle; and detecting a temperature of the solid surface, whereby detection of an increased temperature relative to a temperature of the solid surface that would be detected in the absence of said complex indicates the presence or amount of target nucleic acid sequence hybridized to the solid surface. Thus, in some embodiments, the use of a detection probe is not necessary.

In some embodiments, a nucleic acid hybridization detection assay is carried out at a surface of a solid substrate (e.g., a solid surface). In some embodiments, the solid surface is a solid electrode (e.g., an indium tin oxide electrode), although it is not necessary that the solid surface be an electrode.

The solid surface is modified by single-stranded capture oligonucleotide probes that are immobilized to the surface of the surface. The capture probes hybridize with a complementary target nucleic acid sequence, which in turn is hybridized by a detection probe comprising a nanoparticle. Thus, the target sequence forms part of a hybridization complex comprising a capture probe, a target sequence, and a detection probe.

As used herein, the terms “complex”, “duplex,” and “hybridization complex” are used interchangeably, and mean a structure formed of at least two different members. Hybridization complexes can comprise two or more DNA sequences, RNA sequences or combinations thereof. Complexes, in general, form via hybridization of complementary strands (e.g. by Watson Crick or Hoogsteen base-pairing) of nucleic acids (e.g. DNA or RNA). A member of a hybridization complex can itself comprise one, two or more members. Thus a complex can comprise a structure comprising two members, one or both of which can itself be a complex. For example, one member of a complex can comprise a single stranded nucleic acid sequence (immobilized or in solution) and the second member of the complex can comprise a nucleic acid double stranded complex (immobilized or in solution), effectively making the complex a triplex structure.

The term “target sequence,” as used herein, means a nucleic acid sequence on a single strand of nucleic acid. A target sequence may accordingly be a single-stranded segment of a target nucleic acid. If the target nucleic acid is single-stranded, the target sequence can be identical to the target nucleic acid, or may comprise a portion or sub-sequence of the target nucleic acid. If the target nucleic acid is double-stranded DNA, the target sequence may be identical to or comprise a sub-sequence of the coding strand, or may be identical to or comprise a sub-sequence of the anti-parallel, complementary, non-coding strand. As described in further detail below, target sequences may optionally comprise moieties such as labels or tags that facilitate specific binding to a detection probe comprising a nanoparticle.

A “capture probe,” as used herein, is an oligonucleotide that binds (i.e., hybridizes) to a target nucleic acid sequence, and which is used to probe for the presence of the target sequence. The capture probe enables the attachment of a target nucleic acid to the solid surface, for the purposes of detection. A “detection probe,” as used herein, comprises a nanoparticle. A “detection probe,” as used herein, comprises a nanoparticle. In some embodiments, a detection probe comprises a nanoparticle-oligonucleotide conjugate. Thus, each probe typically comprises an oligonucleotide sequence attached to either a particle or a solid surface. In general, the capture probe is bound to a solid surface, while the detection probe comprises an oligonucleotide attached to a nanoparticle. In some embodiments, a detection probe comprises one partner of a ligand-binding pair (e.g., streptavidin) instead of an oligonucleotide. In some embodiments, a detection probe thus comprises a nanoparticle attached to one partner of a ligand-binding pair.

Nanoparticles and solid substrate surfaces of the present invention may be fabricated from a broad range of materials, although it is preferable that the nanoparticle material and the substrate material are not identical. Moreover, the nanoparticle comprises a material that absorbs light at one or more particular frequencies (e.g., exhibits surface plasmon resonance or interband transition).

In some embodiments, the capture oligonucleotide probe hybridizes a first domain of the target sequence, while the oligonucleotide component of the detection probe hybridizes a second domain of the target sequence to form a hybridization complex. In other embodiments, the detection probe can bind to the same domain as the capture probe, forming a triplex.

Detection of the hybridization complex is facilitated by exposing the solid surface, with hybridization complexes attached, to light being generated (e.g., by a laser) at a wavelength absorbed by the detection probe nanoparticle. Light exposure is carried out subsequent to the formation of the sandwich hybridization complex. Matching the wavelength of light used to photoexcite the nanoparticle with a wavelength absorbed by the nanoparticle generates heat, and elicits a temperature jump in the environment immediately surrounding the nanoparticle and the hybridization complex.

In some embodiments, the wavelength of light used to photoexcite the nanoparticle matches the surface plasmon resonance of the nanoparticle, thus generating heat and eliciting a temperature jump in the environment immediately surrounding the nanoparticle and the hybridization complex. In some embodiments, the wavelength of light does not match the surface plasmon resonance of the nanoparticle, but nonetheless is absorbed by the nanoparticle (e.g., due to interband transition of a metal nanoparticle), thus generating heat.

Comparing the difference between the temperature of the solid surface at the attachment point of the irradiated hybridization complex and the bare solid surface (i.e., the surface unmodified by nanoparticles, also referred to as “background”) provides a measure of nucleic acid hybridization at the surface. This measured temperature, expressed as an absolute temperature or as a temperature difference from background, can be correlated to the concentration of target nucleic acid in the sample. According to experimental results described further below, sensitivities on the order of about 10 fM have been demonstrated.

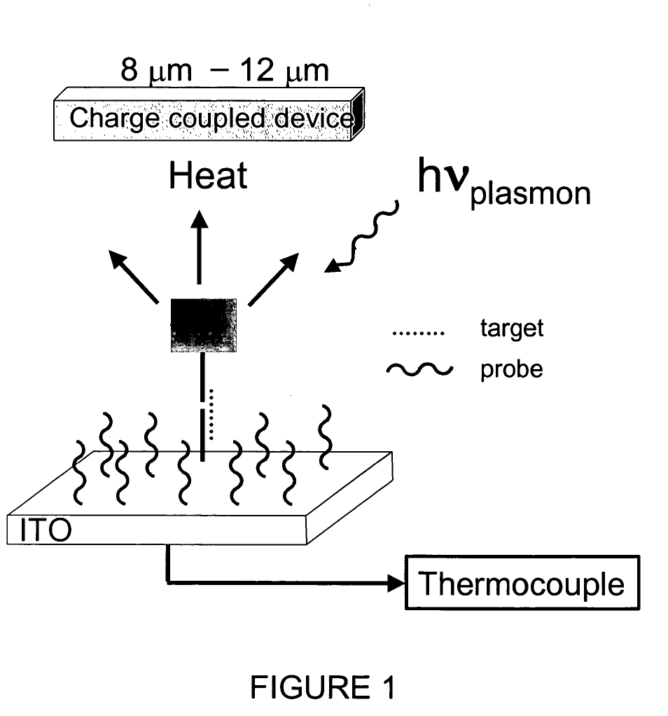

FIG. 1 illustrates a solid surface (here, an indium tin oxide electrode) to which a plurality of capture probes is attached. Target nucleic acid sequence (illustrated as a dotted line) is hybridized to a capture probe, and a detection probe comprising a nanoparticle and an oligonucleotide is hybridized to the target. A hybridization complex comprising a capture probe, target sequence and detection probe is thus illustrated. The hybridization complex is exposed to light energy at a wavelength that is absorbed by the nanoparticle. This irradiation and absorption elicits heat (i.e., a temperature jump) in the immediate environment of the nanoparticle, which can be detected by a thermocouple attached to the back of the solid surface (as shown), or can be detected by a charge coupled device (also shown).

B. Nucleic Acid Sequences

The methods described herein are useful for the detection of target nucleic acid sequences and nucleic acid hybridization events. Probes useful in the detection of target sequences and nucleic acid hybridization events can also comprise nucleic acids, generally in the form of oligonucleotides.

As used herein, the terms “nucleic acid,” “nucleic acid sequence,” “nucleic acid molecule,” and grammatical equivalents mean at least two nucleotides covalently linked together. Nucleic acids may be single-stranded or double-stranded, as specified, or contain portions of both double-stranded or single-stranded sequence. Nucleic acids can comprise any combination of deoxyribo- and ribonucleotides, and any combination of bases, including uracil, adenine, thymine, cytosine, guanine, inosine, xathanine hypoxathanine, isocytosine, isoguanine, etc. Deoxyribonucleic acids (DNA) can comprise genomic DNA, cDNA derived from ribonucleic acid, DNA from an organelle (e.g., mitochondrial DNA or chloroplast DNA), synthesized DNA (e.g., oligonucleotides), or combinations thereof. Ribonucleic acids (RNA) can comprise genomic RNA (e.g., viral genomic RNA), messenger RNA (mRNA), ribosomal RNA (rRNA), transfer RNA (tRNA), or combinations thereof.

A nucleic acid of the present invention will generally contain phosphodiester bonds, although in some cases, as outlined below, nucleic acid analogs are included that may have alternate backbones, comprising, for example, phosphoramide, phosphorodithioate, methylphophoroamidite linkages, and peptide nucleic acid backbones and linkages. Other analog nucleic acids include those with positively-charged backbones, non-ionic backbones and nonribose backbones. Nucleic acids containing one or more carbocyclic sugars are also included within the definition of nucleic acids. Mixtures of naturally occurring nucleic acids and analogs can be used. Mixtures or chimeras of different nucleic acid analogs, and mixtures of naturally occurring nucleic acids and analogs can also be used.

Peptide nucleic acids (PNA) are specifically included in the definition of nucleic acids, as used herein. PNAs are DNA analogs wherein the backbone of the analog (for example, a sugar backbone in DNA) is a pseudopeptide. A PNA backbone can comprise, for example, a sequence of repeated N-(2-amino-ethyl)-glycine units. A peptide nucleic acid analog reacts as DNA would react in a given environment, and can bind complementary nucleic acid sequences and various proteins. Peptide nucleic acid analogs offer the potential advantage over unmodified DNA of the formation of stronger bonds, due to the neutrally charged peptide backbone of the analogs, and can impart a higher degree of specificity than is achievable by unmodified DNA. These backbones are substantially non-ionic under neutral conditions, in contrast to the highly charged phosphodiester backbone of naturally occurring nucleic acids.

Nucleic acids can also comprise “locked nucleic acids”, also known as LNAs (e.g., WO 98/39352).

When used as oligonucleotide probes, as defined herein, nucleic acids can be analytically pure, as determined by spectrophotometric measurements or by visual inspection following electrophoretic resolution. In some embodiments, nucleic acids that are to be amplified can be analytically pure, although purity is not a requirement. In some embodiments, nucleic acid samples are free of contaminants such as polysaccharides, proteins and inhibitors of enzyme reactions. When an RNA sample is intended for use as probe or target sequence, it is preferably free of DNAase and RNAase. Contaminants and inhibitors can be removed or substantially reduced using resins for DNA extraction or by standard phenol extraction and ethanol precipitation, as is taught in the art.

1. Target Nucleic Acids and Sequences

A target sequence can be selected on the basis of the context in which the present methods are employed. Target sequences can vary widely. For example, desirable target sequences include, but are not limited, to characteristic or unique nucleic acid sequences found in various microbes or mutated DNA that can be used in the diagnosis of diseases, in environmental bioremediation, in the determination of genetic disorders, and in genetic epidemiology. Functional equivalents of known sequences can also be used as target sequences.

The target sequence may be a portion of a gene, a regulatory sequence, genomic DNA, cDNA, RNA including mRNA and rRNA, or others. The target sequence can be a target sequence from a biological sample, as discussed herein, or can be a secondary target such as a product of an amplification reaction. The target sequence can take many forms. For example, a target may be contained within a larger nucleic acid sequence, i.e. all or part of a gene or mRNA, a restriction fragment of a plasmid or genomic DNA, among others. Target nucleic acids can be excised from a larger nucleic acid sample using restriction endonucleases, which sever nucleic acid sequences at known points in a nucleic acid sequence. Excised nucleic acid sequences can be isolated and purified by employing standard techniques. Target sequences can also be prepared by reverse transcription processes. See, e.g., Sambrook et al., Molecular Cloning: A Laboratory Manual (Cold Spring Harbor, New York (1992)).

A target sequence can comprise one or more different target domains. A target domain is a contiguous, partial sequence (i.e., a sub-sequence) of the entire target sequence, and may be any nucleotide length that is shorter than the entire target sequence. In some embodiments, a first target domain of a target sequence hybridizes a capture probe, while a second and different target domain hybridizes an oligonucleotide component of a detection probe. Target domains may be adjacent or separated, as indicated. For example, a first target domain can be directly adjacent (i.e., contiguous) to a second target domain, or the first and second target domains may be separated by an intervening target domain. Assuming a 5′ to 3′ orientation of a target sequence, a first target domain may be located either 5′ to a second target domain, or 3′ to a second domain.

If desired, a target sequence may further comprise an additional moiety such as one partner of a ligand-binding pair, in order to facilitate binding to a detection probe comprising the other partner of the ligand binding pair attached to a nanoparticle. For example, the target sequence may comprise a biotin label or tag, which will facilitate binding to a detection probe comprising a nanoparticle attached to streptavidin. The biotin tag may be incorporated into the target sequence using amplification methods that are analogous to known methods used to incorporate fluorescent labels into target molecules, as set forth in more detail below.

Nucleic acid sequences of any practical length can be used as a target sequence. Generally, a target sequence is between ten and 50 nucleotides in length, and thus target sequences of ten, 15, 20, 25, 30, 35, 40, 45 or more nucleotides can be employed. However, target sequences of any length can be employed in the methods of the present invention, and in some cases may be shorter than ten nucleotides and longer than 50 nucleotides. For example, target sequences may be 60 nucleotides long, 75 nucleotides long, 85 nucleotides long, 100 nucleotides long, 300 nucleotides long, or even longer. If desired by the artisan, a target sequence may be fragmented prior to hybridization steps by using enzymatic, mechanical or other means as known in the art.

In some embodiments, target sequences can be isolated from biological samples, including, but not limited to, bodily fluids (e.g., blood, urine, serum, lymph, saliva, anal and vaginal secretions, perspiration, semen, etc., of virtually any organism); environmental samples (e.g., air, plant, agricultural, water and soil samples); and research samples (i.e. amplification reaction products, purified samples such as purified genomic nucleic acids, and unpurified samples of bacteria, virus, genomic DNA, etc.).

If required, the target nucleic acid can be isolated from source biological samples using known techniques. For example, samples can be collected and concentrated or lysed, as required. Appropriate adjustment of pH, treatment time, lytic conditions and sample modifying reagents can also be made in order to optimize reaction conditions. Such modification techniques are well known to those of skill in the art.

Methods for nucleic acid isolation and purification can comprise simultaneous isolation of, for example, total nucleic acid, or separate and/or sequential isolation of individual nucleic acid types (e.g., genomic DNA, cDNA, organelle DNA, genomic RNA, mRNA, polyA⁺ RNA, rRNA, tRNA) followed by optional combination of multiple nucleic acid types into a single sample.

Methods for nucleic acid isolation can optionally be optimized to promote recovery of pathogen-specific nucleic acids. In some organisms, for example fungi, protozoa, gram-positive bacteria, and acid-fast bacteria, cell lysis and nucleic acid release can be difficult to achieve using general procedures, and therefore a method can be chosen that creates minimal loss of the pathogen subset of the sample.

Semi-automated and automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith (1998) The Scientist 12(14):21-24 and Paladichuk (1999) The Scientist 13(16):20-23.

In some embodiments, a target nucleic acid comprises a double-stranded nucleic acid. Double stranded nucleic acid sequences can be prepared, for example, by isolating a double stranded segment of DNA. Multiple copies of single stranded complementary oligonucleotides can also be synthesized and annealed to one other under appropriate conditions. In order to provide a single-stranded target for hybridization, double-stranded nucleic acids are preferably denatured before hybridization. The term “denaturing” refers to the process by which strands of oligonucleotide duplexes are no longer base-paired by hydrogen bonding and are separated into single-stranded molecules. Methods of denaturation are well known to those skilled in the art and include thermal denaturation and alkaline denaturation.

RNA isolation methods are known to one of skill in the art. See, Albert et al. (1992) J Virol 66:5627-2630; Busch et al. (1992) Transfusion 32:420-425; Hamel et al. (1995) J Clin Microbiol 33:287-291; Herrewegh et al. (1995) J Clin Microbiol 33:684-689; Izraeli et al. (1991) Nuc Acids Res 19:6051; McCaustland et al. (1991) J Virol Methods 35:331-342; Natarajan et al. (1994) PCR Methods Appl 3:346-350; Rupp et al. (1988) BioTechniques 6:56-60; Tanaka et al. (1994) J Gen Virol 75:2691-2698; and Vankerckhoven et al. (1994) J Clin Microbiol 30:750-753.

When mRNA is selected as a target sequence, the methods described herein can enable an assessment of pathogen gene expression. For example, detecting a pathogen in a biological sample can comprise determination of expressed virulence factors, other deleterious agents produced by a pathogen, or biosynthetic enzymes that generate virulence or other harmful pathogen gene products. Such analysis can facilitate distinction between active and latent infection, and indicate severity of an infection.

One of the advantages of the sandwich assay embodiments described herein is that the need to use nucleic acid amplification technology, cell culture, or other methods of selectively amplifying a target nucleic acid sequence are greatly diminished or even eliminated. However, while amplification steps are generally not required, procedures that include amplification prior to carrying out the detection methods of the present invention can be desirable in some cases. Nucleic acid “amplification” generally includes methods such as polymerase chain reaction (PCR), ligation amplification (or ligase chain reaction, LCR) and amplification methods based on the use of the enzyme Q-beta replicase. These methods are well known and widely practiced in the art., and reagents and apparatus for conducting them are commercially available.

Other amplification techniques are known in the art and may be used in conjunction with the detection methods described herein. These methods include random-primed PCR (RP-PCR); linker/adaptor-based DNA amplification; sequence-independent, single-primer amplification (SISPA); whole genome PCR; primer-extension pre-amplification (PEP); transcription-based amplification (variously called self-sustaining sequence replication, nucleic acid sequence-based amplification (NASBA), or transcription-mediated amplification (TMA)), amplified antisense RNA (aRNA); global RNA amplification, and others. See, e.g., Kinzler & Vogelstein (1989) Nuc Acids Res 17(10):3645-3653; Peng et al. (1994) J Clin Pathol 47:605-608); Reyes & Kim (1991) Mol Cell Probes 5:473-481; Van Gelder et al. (1990) Proc Natl Acad Sci USA 87:1663-1667; Wang et al. (2000) Nat Biotech 18(4):457-459; Podzorski et al. in Murray et al., eds., Manual of Clinical Microbiology (American Society for Microbiology, Washington, D.C. (1995) p.130); Zhang et al. (1992) Proc Natl Acad Sci USA 89:5847-5851; and U.S. Pat. No. 6,066,457 to Hampson et al.

In accordance with the methods described herein, any one of the above-mentioned amplification methods or related techniques can be employed to amplify the target nucleic acid sample and/or target sequence, if desired. In addition, such methods can be optimized for amplification of a particular subset of nucleic acid (e.g., genomic DNA versus RNA), and representative optimization criteria and related guidance can be found in the art. See, e.g., Cha & Thilly (1993) PCR Methods Appl 3:S18-S29; Linz et al. (1990) J Clin Chem Clin Biochem 28:5-13; Robertson & Walsh-Weller (1998) Methods Mol Biol 98:121-154; Roux (1995) PCR Methods Appl 4:S185-S194; Williams (1989) BioTechniques 7:762-769; and McPherson et al., PCR 2: A Practical Approach (IRL Press, New York, N.Y. (1995)).

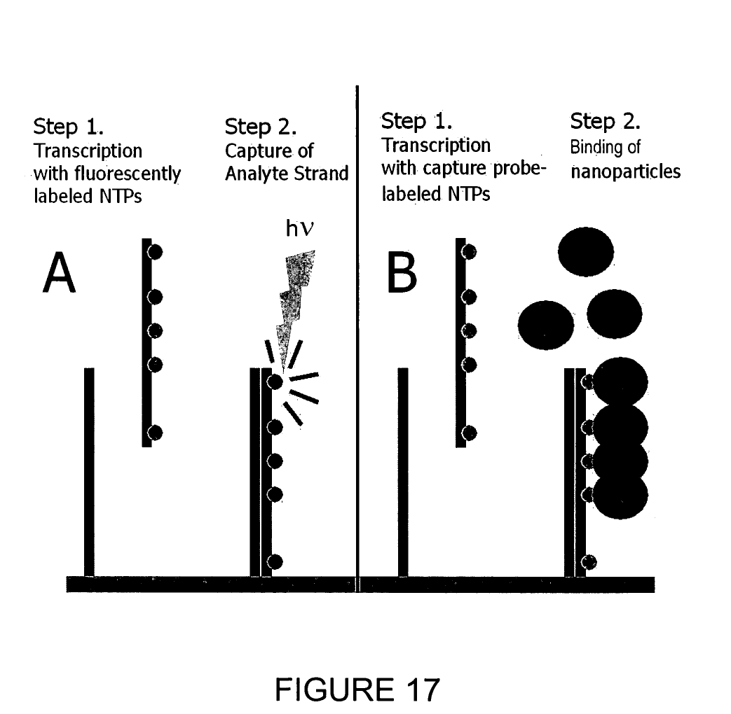

In some embodiments, amplification techniques are used to incorporate labeling or tagging moieties into a target sequence, which moieties are used to facilitate binding to a detection probe. In some embodiments, a target nucleic acid comprises a nucleic acid labeled or tagged with one partner of the ligand-binding pair (e.g., biotin), while a detection probe comprises a nanoparticle attached to the other partner of the ligand-binding pair (e.g., streptavidin). FIGS. 17A and 17B illustrate one method by which a ligand-binding pair moiety such as biotin can be incorporated into a target sequence. FIG. 17A schematically illustrates a known method of incorporating a fluorescent label into a target nucleic acid, in which a target is amplified using fluorescently-labeled nucleotide triphosphates (NTPs). In some embodiments of such a method, a target sequence is, for example, mRNA, and the complement of the target is enzymatically synthesized by means of a reverse transcriptase to produce a fluorescently-labeled cDNA target strand. Upon binding (hybridization) of a detection probe, the hybridization complex is exposed to light and detected by fluorescent detection and imaging means.

FIG. 17B illustrates a method useful in the practice of the present methods, by which biotin-tagged (rather than fluorescently-labeled) NTPs are incorporated into a cDNA target strand, and then used to hybridize nanoparticles coated with streptavidin. Methods of incorporating label and tag moieties (e.g., fluorescent labels, biotin, etc.) into target sequences using transcriptase-based amplification methods are known in the art. See, e.g., U.S. Pat. Nos. 6,589,737; 6,046,038; 6,004,755; 6,203,989; 6,589,742 and 6,503,711.

Thus, in some embodiments, a target sequence incorporates biotin moieties during an amplification reaction in which single stranded (ss) nucleic acid (e.g., mRNA) is used as a template, and nucleotides labeled with biotin are enzymatically incorporated into a complementary cDNA strand using a transcriptase (e.g., reverse transcriptase).

2. Probes

The term “probe,” as used herein, indicates a structure, complex or molecule having a capacity to selectively or substantially hybridize to a complementary target sequence in a heterogeneous mixture of nucleic acid molecules. In some embodiments, probes comprise oligonucleotide molecules. Oligonucleotide probes are typically designed to hybridize to target sequences in order to determine the presence or absence of the target sequence in a sample. As such, oligonucleotide probes as used in the methods described herein are generally designed to be complementary, in whole or in part, to a target sequence, such that hybridization between the target sequence and the probe or probes occurs.

The term “complementary sequences”, as used herein, indicates two nucleotide sequences that comprise antiparallel nucleotide sequences capable of pairing with one another upon formation of hydrogen bonds between base pairs. Additionally, the term “complementary sequences” means nucleotide sequences that are substantially complementary, as can be assessed by hybridization to the nucleic acid segment in question under relatively stringent conditions such as those described herein. The term “complementary sequence” also includes a pair of nucleotides that bind a same target nucleic acid and participate in the formation of a triplex structure as described, for example in U.S. Pat. No. 6,027,893 to Ørum et al. This complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence.

In some embodiments, each probe comprises at least one oligonucleotide sequence, which is complementary to a contiguous nucleic acid sequence of a target sequence such that the oligonucleotide sequence specifically hybridizes to the target sequence under stringent conditions.

The total length of a probe oligonucleotide will vary depending on its use, the length of the target sequence, and the hybridization and wash conditions. In general, oligonucleotide sequences of 5 to 50 nucleotides can be used; however, shorter or longer sequences can, in certain instances, be employed. In some cases, longer probes may be used, e.g. from about 50 to about 200-300 nucleotides or even longer in length.

In some embodiments, single-stranded DNA is used as an oligonucleotide component of the probes used in the present methods. In some embodiments, two oligonucleotides complementary to separate, non-overlapping segments, regions or domain of a target nucleic acid sequence are used in the sandwich hybridization format. In these embodiments, one of the oligonucleotides is used as a capture probe, while the other comprises the oligonucleotide component of the corresponding detection probe. By using two non-overlapping, non-complementary probes to identify a target nucleic acid sequence, the risk of “background noise” being interpreted as a false positive reading is reduced as compared to a system that relies on the hybridization of a single probe for detection.

Methods of making oligonucleotides of a predetermined sequence are well-known. See, e.g., Sambrook et al., supra, and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides. Oligoribonucleotides and oligodeoxyribonucleotides can also be prepared enzymatically.

C. Solid Substrate Materials

Suitable solid substrate material can be selected according to desired thermal absorption range, ease of surface attachment of nucleic acid to surface, and correct or desired optical properties. In general, the solid substrate can be any material that does not absorb significantly at the light excitation wavelength used for nanoparticle irradiation. As provided above, it is preferable that the solid substrate material not be identical to the material of the detection probe nanoparticle.

The solid substrate can be constructed entirely of a single material, or may comprise one material layered over one or more other materials. Solid substrate materials include polymers or otherwise non-conducting surfaces. Thus, the substrate can comprise a wide variety of materials, including but not limited to, carbon and its various forms (including diamond, graphite, carbon paste), glass, fiberglass, teflon, ceramics, silicon, mica, plastic (including acrylics, polystyrene and copolymers of styrene and other materials), polypropylene, polyethylene, polybutylene, PDMS, polycarbonate, polyurethanes, TEFLON™, and combinations thereof. In some embodiments, the solid substrate is a carbon electrode, such as a glassy carbon electrode.

Solid substrates can, but need not necessarily be, solid electrodes. As used herein, the term “electrode” means a composition that is able to carry or sense a current or charge, and then convert it to a measurable signal. Representative electrode materials include, but are not limited to, certain metals and their oxides, such as gold, platinum, palladium, aluminum, indium tin oxide (ITO), tin oxide, fluorine-doped tin oxide, cadmium oxide, iridium oxide, ruthenium oxide, zinc tin oxide, antimony tin oxide; platinum oxide, titanium oxide, palladium oxide, aluminum oxide, molybdenum oxide, tungsten oxide, and others. In some embodiments, the electrode comprises indium tin oxide (ITO). Representative materials that can be used in a support are described in Brewer et al., (2002) J. Phys. Chem. B 106:11446.

Solid substrates, particularly electrodes, can also include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g., all electrodes and interconnections in a plane) or “three dimensional” (wherein the electrodes are on one surface and the interconnects may go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

The solid surfaces described herein are depicted in the Figures as flat surfaces. However, a flat surface is only one of the possible conformations of the solid substrate surfaces, and as such is illustrated for schematic purposes only. The conformation of the surface will vary with the specific detection method used. For example, flat planar surfaces may be preferred for methods requiring addressable locations for detection.

D. Modification Of Solid Surface With Capture Probes

In some embodiments, the solid surface comprises a plurality of capture probes attached to the solid surface in an array format. As used herein, the terms “nucleic acid microarray,” and “nucleic acid hybridization array” are used interchangeably, and mean an arrangement of a plurality of nucleic acid sequences (e.g., capture probes) bound to a solid surface (also referred to herein as a “support”). The terms “addressable array” and “array” are used interchangeably, and mean a plurality of entities arranged on a support in a manner such that each entity occupies a unique and identifiable position. In the methods described herein, the entities are capture probes (e.g., capture oligonucleotides) immobilized to the surface of a solid substrate. As used herein, the terms “immobilize” and “attach” are used interchangeably to mean a chemical and/or mechanical association of one moiety with one or more surfaces. The association can be covalent or non-covalent, and can be direct or indirect.

In some embodiments, capture probes attached to a solid surface are ordered such that each capture probe sample has a unique, identifiable location on the surface. The physical location on the surface where a capture probe is attached or immobilized is referred to herein as an “attachment point.” The identity of a capture probe bound to a surface at a given location can be determined in several ways. One exemplary way to correlate a capture probe with its location is to attach the capture probe to the support at a known position (see, e.g., Pirrung, (1997) Chem. Rev. 97: 473-486). Discrete locations on the support can be identified using a grid coordinate-like system. In this approach, the working area of the support surface can be divided into discrete areas that may be referred to interchangeably as “spots” or “patches”. Different capture probes can subsequently be attached to the surface in an orderly fashion, for example, one capture probe, or one sample of identical capture probes, to a spot. In this strategy, the probe oligomers can be applied one or several at a time. In one exemplary method, sites at which it might be desirable to temporarily block probe binding can be blocked with a blocking agent. The blocking agent can be subsequently removed and the site freed for probe binding. This process can be repeated any number of times, thus facilitating the attachment of a known probe at a known location on a support.

Localizing capture probes to an support surface at known locations can involve the use of microspotting. In this approach, the location of the capture probes on a surface is determined by the ordered application of probe samples in a group. That is, capture probes are ordered in known locations prior to application to the surface. In this way, the location of each probe is known as it is applied. Appropriate devices for carrying out this approach are commercially available and can be used with the detection methods described herein. For example, the present methods are compatible with the commercially available GENECHIP™ system (Affymetrix, Inc., Santa Clara, Calif.) or the commercially available SPOTBOT™ Automated Spotting Arrayer (TeleChem International, Sunnyvale, Calif.).

As set forth above, in some embodiments a single-stranded nucleic acid sequence is used as a capture probe. For example, a capture probe can comprise a single-stranded cDNA sequence complementary to a target gene of interest or to a target domain thereof. The capture probe can be attached to the substrate surface indirectly via an “attachment linker,” as defined herein. In these embodiments, one end of an attachment linker is attached to a capture probe, while the other end (although, as will be appreciated by those in the art, it need not be the exact terminus for either) is attached to the surface.

The method of attachment of the capture probe to the attachment linker can generally be done as known in the art, and will depend on the composition of the attachment linker and the capture probe. In general, the capture probe is attached to the attachment linker through the use of functional groups on each moiety that can then be used for attachment. Preferred functional groups for attachment are amino groups, carboxy groups, oxo groups and thiol groups, although others are used and described in more detail below. Using these functional groups, the capture probes can be attached using functional groups on the solid surface.

In one example of an attachment approach suitable for attachment of capture probes to a solid surface, one or more probe capture sequences are initially incubated with a solution of a thio-alcohol for a pre-selected period of time. In some embodiments, C6 mercaptohexanol is employed as a thio-alcohol, in accordance with techniques described by Loweth et al., (1999) Angew. Chem. Int. Edit. 38: 1808-12, and Storhoff & Mirkin, (1999) Chem. Rev. 99: 1849-62. Thio-alcohol and capture probe are added in amounts so as to bring the final concentration of capture probe in the solution to about 20% or less. The incubation time permits the covalent association of the 3′ end of the capture probe oligonucleotide with the hydroxyl group of the thio-alcohol. The solution is then exposed to the surface of a support under conditions that permit association of the sulfur atom of the thio group with the surface of the support. Suitable equipment is commercially available and can be used to assist in the binding of a target sequence to a support surface.

In another specific example, a monolayer of 12-phosphonododecanoic acid is formed on the solid surface. The carboxylic acid of 12-phosphonododecanoic acid is then activated by 1-ethyl-3-(3-dimethylaminopropyl)carbodiimide hydrochloride (EDC) to form an O-acylisourea intermediate. See, e.g., S. H. Brewer et al., Langmuir (2002) 18, 6857-6865; B. L. Frey and R. M. Corn, Analytical Chemistry (1996) 68, 3187-3193; M. Burgener et al., Bioconjugate Chemistry (2000) 11, 749-754; K Kerman et al., Analytica Chimica Acta (2002) 462, 39-47; E. Huang et al., Langmuir (2000) 16, 3272-3280; and G. T. Hermanson, Bioconjugate Techniques (1996) (Academic Press: San Diego). This activated carboxylic acid group is attacked by the primary amine (acting as a nucleophile) of a 5′-modified C₃NH₂ single-stranded DNA strand to form an amide bond between the monolayer of 12-phosphonododecanoic acid and the 5′ modified C₃NH₂ ssDNA.

Other functional groups useful for attaching oligonucleotides to solid surfaces (including nanoparticles) include, for example, moieties comprising thiols, carboxylates, hydroxyls, amines, hydrazines, esters, amides, halides, vinyl groups, vinyl carboxylates, phosphates, silicon-containing organic compounds, and their derivatives. Other functional groups useful for attachment include phosphorothioate groups (see, e.g., U.S. Pat. No. 5,472,881 for the binding of oligonucleotide-phosphorothioates to gold surfaces), aminosilanes (see, e.g,. K. C. Grabar et al., J. Am. Chem. Soc.(1996) 118, 1148), and substituted alkylsiloxanes (see, e.g. Burwell, Chemical Technology 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides terminated with a 5′ thionucleoside or a 3′ thionucleoside can also be used for attaching oligonucleotides to solid surfaces.

In some embodiments, a “tag” or “linker” nucleic acid sequence can be employed to attach capture probes to solid surfaces. When a tag sequence is employed, an surface can comprise a tag nucleic acid complement. A tag complement is a sequence that is complementary to a tag sequence associated with a capture probe. Thus, when a capture probe comprising a tag sequence is contacted with an surface comprising a tag complement under suitable hybridization conditions, a duplex can form.

A tag sequence can comprise, for example, a sequence that is complementary to a support-bound tag complement. A tag sequence can be associated with a target sequence, which can then be amplified by PCR prior to association with a nanoparticle. The PCR amplicon will comprise a nucleic acid sequence comprising the tag sequence and a target target sequence. The PCR amplicon then comprises a sequence that is complementary to a support-bound tag complement. Inclusion of a tag sequence, for example as a component of a target sequence, offers the advantage that a support need not be specific for a given target sequence, but rather can be universal in the sense that it is specific for a tag complement, but not for any particular target sequence. Thus, by employing a tag complement, a solid surface (or nanoparticle, as described herein) can be independent of the source of a capture probe oligonucleotide (as to species, etc.) in the sense that the surface can be specific for a tag sequence, but not for any particular capture probe sequence. Thus, by employing a method comprising the use of a tag-tag complement approach, the need to form different supports for different probe and/or target sequences is mitigated. See, e.g., WO 94/21820, WO 97/31256, WO 96/41011 and U.S. Pat. No. 5,503,980.

E. Nanoparticle Components

Detection probes used in the practice of the presently described methods generally comprise at least two components. In some embodiments, the two components include an oligonucleotide nucleic acid sequence, and a nanoparticle to which the oligonucleotide is attached.

In some embodiments, a non-oligonucleotide ligand is used instead of an oligonucleotide sequence. In some embodiments, the non-oligonucleotide ligand is a member of a ligand-binding pair, and its other, corresponding member of the binding pair is attached to or incorporated into the target sequence, such that the target sequence can specifically or selectively bind the detection probe. In one example of these embodiments, a target sequence is biotinylated according to methods described above (e.g., nucleic acid amplification incorporating biotin-tagged nucleotides), while a detection probe comprises a nanoparticle coated with streptavidin. Methods for attaching streptavidin to nanoparticles are known, see, e.g., Shaiu et al., Nuc. Acids Res. 21, 99 (1993).

Detection probes may also and optionally comprise other useful moieties, including catalysts, supplementary labeling molecules (e.g., fluorescent, magnetic or chemiluminescent moieties), detection enhancers, and the like.

As used herein, the terms “nano”, “nanoscopic”, “nanometer-sized”, “nanostructured”, “nanoscale”, and grammatical derivatives thereof are used synonymously, and in some cases interchangeably. As used herein, the term “nanoparticle” means any structure comprising a nanoparticle and can include a component to which a nucleic acid is bound. Typically, but not necessarily, a nanoparticle is an approximately spherical metal atom-comprising entity. In one example, a nanoparticle is a particle comprising a material such as a metal, a metal oxide or a semiconductor. In other examples, a nanoparticle can comprise a polymeric species or any other conducting material.

Nanoparticles are generally less than about 1000 nanometers (nm) in diameter, usually less than about 200 nanometers in diameter and more usually less than about 100 nanometers in diameter. In certain particular embodiments, nanoparticles are between about 10 nm and 20 nm in diameter, while in other embodiments, the size of the nanoparticle is less than about 10 nm. Representative ranges of nanoparticle sizes include but are not limited to from about 5 to about 200 nanometers, from about 5 to about 100 nanometers, from about 5 to about 50 nanometers, from about 5 to 20 nanometers, from about 10 to about 200 nanometers, from about 10 to about 100 nanometers, and from about 10 to about 50 nanometers.

A nanoparticle can comprise almost any material, as long as the material is able to absorb light at a known wavelength. In some embodiments, the nanoparticle will comprise a material known to exhibit surface plasmon resonance. The skilled artisan will be able to readily determine whether a putative nanoparticle material exhibits surface plasmon resonance, either because this characteristic of the material is known in the art, or because it can be determined by methods known in the art. See, e.g., B. Liedberg et al., Biosens. Bioelectron. (1995) 10: i-ix, and J. Homola et al., Sensors and Actuators B (1999) 54: 3-15. In other embodiments, the nanoparticle material comprises metal and exhibits interband transitions.

Considerations when selecting and/or designing a nanoparticle can include size, material, thermal reactivity of the material, the ease with which an oligonucleotides can associate with the nanoparticle. Additionally, it can be desirable to associate an additional, secondary component with a nanoparticle. Exemplary secondary components include, but are not limited to, catalysts, supplementary labeling molecules (e.g., fluorescent, magnetic or chemiluminescent moieties), and detection enhancers. Therefore, the reactivity of a nanoparticle to a desired secondary component can also be a consideration. Thus, considerations when selecting and/or designing a nanoparticle can include size, material, chemical reactivity of the material the ease with which an oligonucleotides can associate with the nanoparticle, and the ease with which a secondary component can associate with the nanoparticle.

Nanoparticles can be formed from metals and metal oxides, including but not limited to gold, silver, titanium, titanium dioxide, tin, tin oxide, iron, iron^{III} oxide, copper, nickel, aluminum, steel, indium, platinum, indium tin oxide, fluoride-doped tin, ruthenium oxide, germanium cadmium selenide, cadmium sulfide and titanium alloy. Nanoparticles can also be formed from semiconductor materials (e.g., CdSe, CdS, and CdS or CdSe coated with ZnS) and magnetic (e.g., ferromagnetite) colloidal materials.

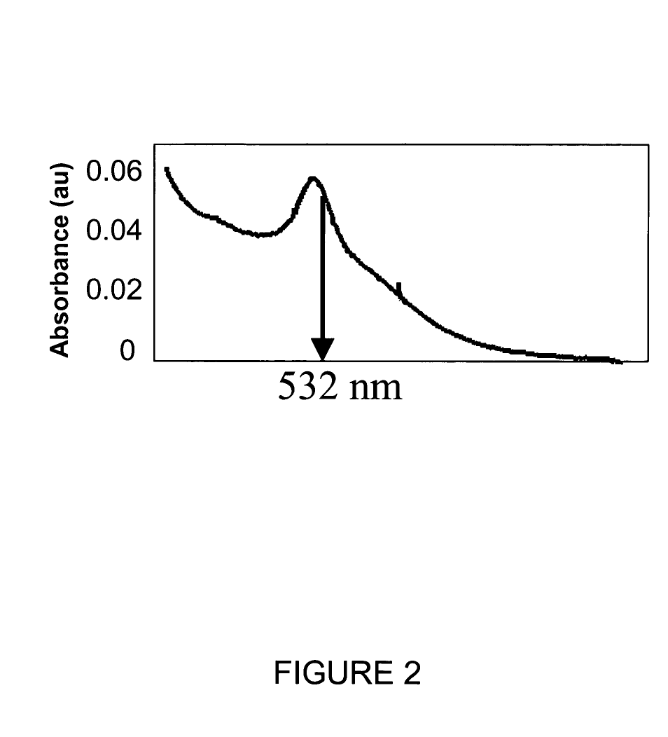

In some embodiments, the nanoparticle material is comprises one or more of gold, silver and platinum, or a combination alloy of any of the foregoing. In some embodiments, the nanoparticle comprises gold. See FIG. 2, which illustrates an absorbance spectrum of a gold nanoparticle as a function of increasing light wavelength. When the gold nanoparticle is irradiated at 532 nm (near the calculated surface plasmon resonance of the gold nanoparticle), a jump in absorbance is observed. Similar behavior is exhibited by silver nanoparticles when irradiated with a wavelength in the 420-460 nm range. As used herein, the term “gold” means element 79, which has the chemical symbol Au; “silver” means element 47, which has the chemical symbol Ag, and “platinum” means element 78, which has the chemical symbol Pt.

Nanoparticles comprising the above-listed materials are generally available commercially from numerous suppliers, including but not limited to Vacuum Metallurgical Co., Ltd. (Chiba, Japan), Vector Laboratories, Inc. (Burlingame, Calif.), Ted Pella, Inc., Amersham Corporation and Nanoprobes, Inc.

Nanoparticles can be fabricated using any suitable method, if desired. See, e.g., Marinakos et al. (1999) Adv. Mater. 11:34; Marinakos et al. (1998) Chem. Mater. 10:1214-19; Enustun & Turkevich (1963) J. Am. Chem. Soc. 85:3317; Hayashi (1987) J. Vac. Sci. Technol. A5(4): 1375-84; Hayashi (1987) Phys. Today, December 1987, 44-60; MRS Bulletin, January 1990, pp. 16-47; G. Schmid, (ed.) Clusters and Colloids (V C H, Weinheim, 1994); M. A. Hayat (ed.) Colloidal Gold: Principles, Methods, and Applications (Academic Press, San Diego, 1991); R. Massart, IEEE Transactions On Magnetics, 17, 1247 (1981); T. S. Ahmadi, et al., Science, 272, 1924 (1996); A. Henglein, et al., J. Phys. Chem., 99, 14129 (1995); A. C. Curtis. et al., Angew. Chem. Int. Ed. Engl., 27, 1530 (1988); Weller, Angew. Chem. Int. Ed. Engl., 32, 41 (1993); Henglein, Top. Curr. Chem., 143, 113 (1988); Henglein, Chem. Rev., 89, 1861 (1989); Brus. Appl. Phys. A., 53, 465 (1991); Bahncmann, in Photochemical Conversion and Storage of Solar Energy (eds. Pelizetti and Schiavello 1991), page 251, and others.

Special metal coated particles known as “nanoshells” are also included in the definition of the term “nanoparticles,” in the practice of the present methods. In general, nanoshells comprise a non-conducting, semiconductor or dielectric core coated with an ultrathin metallic layer. In general, nanoshells have diameters ranging from a few nanometers up to about 5 microns, and have defined wavelength absorbance maxima across the visible and infrared range of the electromagnetic spectrum. Gold nanoshells are one class of optically active nanoparticles that consist of a thin layer of gold surrounding a dielectric core, such as gold sulfide (see, e.g., R. D. Averitt et al., J. Opt. Soc. Am. B 16:1824-1832 (1999), and R. D. Averitt et al., Phys. Rev. Lett. 78:4217-4220 (1997)), or other materials.

Metal nanoshells possess optical properties similar to metal colloids, i.e., a strong optical absorption and an extremely large and fast third-order nonlinear optical (NLO) polarizability associated with their plasmon resonance. The plasmon resonance frequency of metal nanoshells depends on the relative size of the nanoparticle core and the thickness of the metallic shell (see, e.g., A. E. Neeves, et al. J. Opt. Soc. Am. B 6:787 (1989) and U. Kreibig, et al., Optical Properties of Metal Clusters (Springer, New York (1995)). By adjusting the relative core and shell thickness, metal nanoshells can be fabricated that will absorb or scatter light at any wavelength across the entire visible and infrared range of the electromagnetic spectrum. The relative size or depth of the particle's constituent layers determines the wavelength of its absorption. Whether the particle absorbs or scatters incident radiation depends on the ratio of the particle diameter to the wavelength of the incident light.

For any given core and shell materials, the maximum absorbance of the particle depends upon the ratio of the thickness (i.e., radius) of the core to the thickness of the shell. Based on the core radius:shell thickness (core:shell) ratios that are achieved by the referenced synthesis method, nanoshells manifesting plasmon resonances extending from the visible region to approximately 5 μm in the infrared can be readily fabricated. By varying the conditions of the metal deposition reaction, the ratio of the thickness of the metal shell to the core radius can be varied in a predictable and controlled way. Accordingly, particles are constructed with core radius to shell thick ratios ranging from about 2-1000. This large ratio range coupled with control over the core size results in a particle that has a large, frequency-agile absorbance over most of the visible and infrared regions of the spectrum.

The nonlinear optical (NLO) properties of metal nanoshells or nanoshells-constituent materials can be resonantly enhanced by judicious placement of the plasmon resonance at or near the optical wavelengths of interest. The extremely agile “tunability” of the plasmon resonance is a property particular to metal nanoshells. The resonance of the optical absorption and NLO properties of a nanoshell can thus be systematically designed.

F. Attachment Of Binding Partners To Nanoparticles

The methods described above in reference to attaching oligonucleotides to solid substrate surfaces can also be used to attach oligonucleotides to nanoparticle components of detection probes. For instance, oligonucleotides functionalized with alkanethiols at their 3′-termini or 5′-termini readily attach to gold nanoparticles. See, e.g., Whitesides, Proceedings of the Robert A. Welch Foundation 39th Conference On Chemical Research Nanophase Chemistry, Houston, Tex., pp. 109-121 (1995); Mucic et al., Chem. Commun. (1996) 555-557.

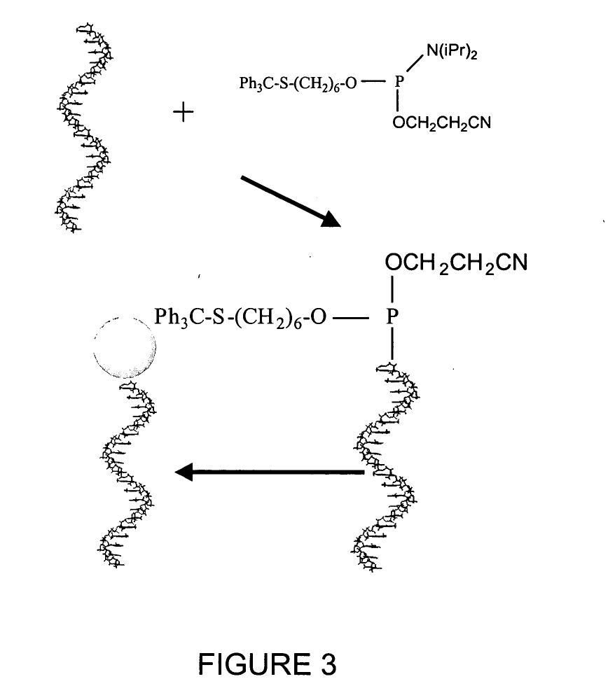

When attaching an oligonucleotide probe to a nanoparticle, a thiolation reaction can be performed to add a thiol group to the 5′ end of a single-stranded oligonucleotide. An amination reaction can be performed and will proceed mutatis mutandis with the thiolation reaction described herein. The general purpose of the reaction is to introduce a nucleophilic center that can subsequently be functionalized with a nanoparticle as described herein. As shown in FIG. 3 and immediately below, a suitable thiol modifier phosphoramidite reagent is the following compound, which is available from Glen Research, Corp. of Sterling, Va.:

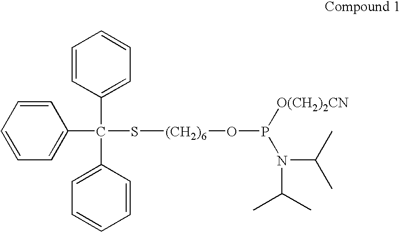

Referring now to FIG. 3, single-stranded oligonucleotides are incubated with a thiol modifier phosphoramidite under anhydrous conditions that permit attachment of the phosphine to the 5′ end of the oligonucleotide. The reaction can be carried out in a nucleic acid synthesizer under standard (and anhydrous) conditions. The thiol modifier is generally added in the last step of synthesis of an oligonucleotide. The phosphine is oxidized using iodine, and the purification is generally the same as that used for unlabeled oligonucleotides. In this reaction, the thiol group is generally protected by a protecting trityl or acetic thioester group and is separated from the 5′-phosphodiester by a variable-length carbon linker. A six-carbon linker is represented in the structure of Compound 1.

The oligonucleotide complex is then subjected to thiol deprotection to remove the trityl group. Specifically, the protecting trityl group is removed by treatment with silver nitrate and dithiothreitol (DTT). The oligonucleotide complex is then incubated with a nanoparticle. The two entities are joined at the thiol exposed by the removal of the trityl group during the deprotection reaction. The formed nanoparticle-oligonucleotide conjugates (i.e., detection probes) can be maintained in the reaction vessel until use.

When a non-synthetic (i.e. isolated, extended or reverse transcribed) oligonucleotide is employed as a component of the detection probe in the present invention, the oligonucleotide can be attached to a nanoparticle in a variety of ways. One mechanism for attaching a non-synthetic oligonucleotide probe to a nanoparticle, generally described as an “end-labeling” scheme, involves derivatizing the 5′ hydroxyl group of an oligonucleotide to incorporate a functional group reactive with the nanoparticle material on the 5′ end of the oligonucleotide. A representative, but non-limiting, list of functional groups includes carboxylate groups, amine groups and thiols group. Such functional groups can be added to an oligonucleotide as a step in the synthesis of the oligo and can be programmed as an additional step in automated nucleic acid synthesizers, as is known in the art.

In some embodiments of an attachment scheme, an oligonucleotide having a 5′ hydroxyl group is incubated under suitable anhydrous reaction conditions with N,N′ carbonyldiimidazole and subsequently with a cysteamine, thereby end labeling the oligo with a thiol group according to Reaction Scheme 1:

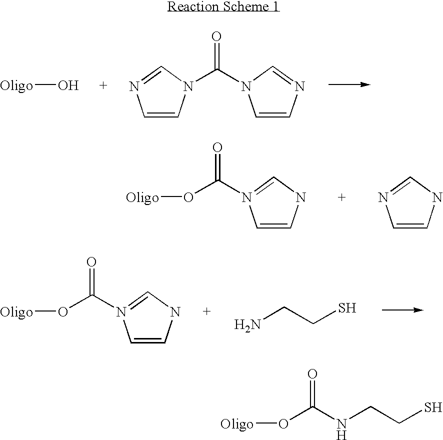

In some embodiments of an attachment scheme, a carboxylate (or a thiol, amine or any other moiety) moiety can be chemically incorporated into a reverse transcription reaction or, as noted, attached to the 5′ hydroxyl of a synthesized oligonucleotide. Similarly, phosphonates and amines can be employed to attach an oligonucleotide to a metal oxide or a nanoparticle. Cystamine-based attachment strategies can also be employed. Those of ordinary skill in the art can recognize reaction conditions that might be damaging to an oligonucleotide and can design attachment strategies, using the above disclosure as a guide, so as to maintain the integrity of the oligonucleotide. It is noted that a deoxynucleotide phosphate (dNTP) having a 5′ hydroxyl group can also be derivatized using Reaction Scheme 1 for attachment to a nanoparticle. Suitable protective groups and additional reaction conditions can be employed, and are known to those of skill in the art.

Although the examples provided above illustrate the attachment of one moiety (i.e., an oligonucleotide) to one nanoparticle, the present methods specifically encompass embodiments in which a plurality of moieties are attached to a single nanoparticle (i.e., the nanoparticles of the present methods are polyvalent). In some embodiments, a plurality of identical oligonucleotides is attached to one nanoparticle. In some embodiments, one or more identical oligonucleotide sequences are attached to the nanoparticle, as well as one or more other, non-oligonucleotide embodiments (e.g., one or more labels, tags, ligand-binding pair components, etc., as previously described herein). Alternatively or in addition, and as explained above, nanoparticles can be attached to non-oligonucleotide components entirely, as in the case of a detection probe comprising a nanoparticle attached to ligand-binding pair components such as streptavidin.

G. Sandwich Format Hybridization Assays

After a capture probe has been immobilized to a solid surface, a target nucleic acid has been selected and a detection probe comprising a nanoparticle has been prepared, a series of hybridization reactions are performed. In some embodiments, the hybridization reactions are carried out in a sandwich assay format. Generally, a target sequence is brought into contact with an solid substrate whose surface has been modified by attaching capture probes to the surface. The target sequence may be brought into contact with the capture probe under hybridization conditions in any suitable manner. In some embodiments, the target sequence is in solution, and the surface having the capture probe immobilized thereon is immersed into the solution containing the target sample. In some embodiments, the solution is a biological sample.

If the capture oligonucleotide and the target nucleic acid comprise complementary sequences, the target sequence will hybridize with the capture probe, thus forming a first hybridization complex comprising a capture probe and a target sequence. After capture and target nucleic acids have been permitted to hybridize, any unbound (unhybridized) nucleic acid can be removed from the solid surface after the hybridization reaction.

In some embodiments, the capture probes attached to the surface have sequence complementary to a first domain of the target sequence to be detected. The target sequence is contacted with the capture probe under conditions effective to allow hybridization of the capture probe with the target. In this manner, the target becomes bound to the capture probe. Any unbound target sequence can optionally be removed from the surface before adding detection probe, as defined herein.

To complete the sandwich assay, the solid surface (with capture probe-target sequence hybridization complexes attached thereto) is brought into contact under hybridization conditions with a detection probe sample comprising nanoparticle-oligonucleotide conjugates. The detection probe may be present in a solution, which can be dispensed onto the solid surface. Alternatively or in addition, the solid surface can be immersed into the solution comprising the detection probe. If a first hybridization complex has formed at a location on the surface, the oligonucleotide component of the detection probe will hybridize to the target sequence component of the first hybridization complex, thus forming a second hybridization complex comprising a capture probe, a target sequence and a detection probe comprising a nanoparticle, which second hybridization complex is attached to the surface by means of the capture probe. The hybridization steps can be performed in any order, or simultaneously, with or without intervening wash steps.

In some embodiments, the oligonucleotide component of the detection probe has sequence complementary to a second domain of the target nucleic acid, and the contacting takes place under conditions effective to allow hybridization of the oligonucleotides attached to the nanoparticle to the target sequence. In this manner, the detection probe nucleic acid-nanoparticle conjugates become attached to the as part of a hybridization complex. After the detection probe has been hybridized to the target, unbound nanoparticle-oligonucleotide conjugates and can be removed from the surface.

Thus, the methods described herein utilize capture and detection probes that substantially hybridize to a target sequence. The phrases “hybridizing substantially to” and “substantially hybridizes” refer to complementary hybridization between a probe nucleic acid molecule and a target nucleic acid molecule, and embraces hybridization of substantially identical sequences that can be accommodated by adjusting the stringency of the hybridization media to achieve the desired hybridization.

The terms “specifically hybridizes” and “selectively hybridizes” each refer to binding, duplexing, or hybridizing of a molecule only to a particular nucleotide sequence under stringent conditions when that sequence is present in a complex or heterogeneous nucleic acid mixture.

An extensive guide to the hybridization of nucleic acids is found in Tijssen, Laboratory Techniques in Biochemistry and Molecular Biology-Hybridization with Nucleic Acid Probes (Elsevier, New York, N.Y. (1993) Part I, Chapter 2). A variety of hybridization conditions may be used in the present invention, including high, moderate and low stringency conditions; see for example Maniatis et al., supra, and Ausubel, et al., supra. The hybridization conditions may also vary when a non-ionic backbone, i.e. PNA is used, as is known in the art.

Stringent conditions are those that allow hybridization between two nucleic acid sequences with a high degree of homology, but preclude hybridization of random, non-complementary sequences. In general, hybridization at low temperature and/or high ionic strength is termed low stringency, and hybridization at high temperature and/or low ionic strength is termed high stringency. The temperature and ionic strength of a desired stringency are understood to be applicable to particular lengths of nucleic acid sequences, to the base content of the sequences, and to the presence of other compounds such as formamide in the hybridization mixture.

Stated otherwise, “stringent hybridization conditions” and “stringent hybridization wash conditions,” in the context of nucleic acid hybridization experiments, are both sequence- and environment-dependent. In general, longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

One can employ varying conditions of hybridization to achieve varying degrees of selectivity of probe towards target sequence, with the general rule that the temperature remain within approximately 10° C. of the duplex's predicted Tₘ, which is the temperature (under defined ionic strength and pH) at which 50% of the target sequence hybridizes to a perfectly matched probe. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. A high stringency wash can optionally be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C.

For shorter sequences (e.g., about 10 to 50 nucleotides), stringent conditions typically involve incubation in salt concentrations of less than about 1.0 M sodium ion, typically about 0.01 to 1.0 M sodium ion (or other ion) concentration, at pH 7.0-8.3, at a temperature of at least about 30° C.

For applications requiring high selectivity, one will typically desire to employ relatively stringent conditions to form hybridization complexes, e.g., conditions of high stringency where one will select relatively low salt and/or high temperature conditions, such as provided by about 0.02 M to about 0.15 M NaCl at temperatures of about 50° C. to about 70° C. Such selective conditions tolerate little, if any, mismatch between the probe and the target strand.

It is generally appreciated that conditions can be rendered more stringent by the addition of increasing amounts of formamide, which serves to destabilize the hybridization complex in the same manner as increased temperature. Thus, hybridization conditions can be readily manipulated, and conditions can be readily selected depending on the desired results.

H. Detection of Hybridization Reaction

As used herein, the term “detect” means determining the presence of a target molecule, entity or event. Determination is carried out by observing the occurrence of a detectable signal (e.g., an electrical, chemical, visual or spectroscopic signal) that occurs in the presence of the target molecule or entity, or during the occurrence of the target event (i.e., a hybridization event). Determination can be qualitative (i.e., detecting mere presence or absence, or detecting relative amounts), or can be quantitative (i.e., specific amounts are measured or quantitated).

After formation of hybridization complexes, the solid surface is exposed to light, as explained in more detail below. The light elicits heating and a temperature jump in the immediate environment of an attached nanoparticle and its hybridization complex. No temperature jump is generated by non-hybridized capture probes, because these capture probes are not also attached to a detection probe comprising a nanoparticle. The thermal event (i.e., generation of heat, temperature jump) can be measured In some embodiments, the generated electrical currents is measured for each either for the entire nucleic acid-modified surface or by hybridization complex attachment point individually, and compared to the background temperature of the bare solid substrate surface (i.e., the surface with no nanoparticles attached thereto).

The detected signal can also be compared to a predetermined threshold or control. The control can be any appropriate control, such as a control under substantially the same conditions, except that no nucleic acids are present, or only non-target sequences are present.

In some embodiments, a competitive assay format is provided. Unlabeled sample target sequences compete with a predetermined amount of competitive, labeled sequences, for hybridizing to capture probes.

Thermal detection can be achieved by irradiating the solid surfaces individually with a light source. Light sources can comprise, for example, a tungsten halogen light source, a xenon arc lamp or a laser (e.g., a YAG laser). In some embodiments, the exposing is by rastering, and the exposing and the detecting are performed simultaneously, as set forth in more detail below.

In some embodiments, a light source can be configured so as to allow irradiation of samples individually and sequentially, for example when a plurality of samples is being scanned. When a laser is used, the beam can be rastered across the support in a predictable pattern, such as horizontally or vertically. The rastering motion can be staggered so as to permit irradiation and detection of a current carried by a given sample (e.g. at an attachment point at which a nanoparticle-comprising hybridization complex was formed), before a subsequent (e.g. sequential) sample is irradiated and monitored for the presence of a current. In one example, a light source, such as a rastering laser beam, can be used to irradiate discrete points on the support, correlating to the attachment points of the capture probes on the support. If a nanoparticle is attached to an attachment point, irradiation generates a temperature jump that is detected by monitoring the temperature through or at the solid support. Each array attachment point (and therefore each potential site of hybridization complex formation) is irradiated, and any generated temperature jump detected, in a sequential fashion, as can be accomplished through the use of a rastering light source.

The artisan can select the wavelength at which to irradiate the nanoparticles attached to the surface based on the material comprising the nanoparticle or nanoshell. As provided above, in some embodiments the wavelength of light matches the surface plasmon resonance of the material that comprises the nanoparticle. In some embodiments, the wavelength of the irradiation matches another wavelength that is absorbed by the nanoparticle (e.g., a wavelength at which a nanoparticle metal component undergoes interband transition). By “matching” is meant that the wavelength of light is identical to (i.e., equivalent) or is nearly equivalent to a light wavelength known to be absorbed by the nanoparticle, which absorbance causes the nanoparticle to generate heat. The particular wavelength will be determined by the material comprising the nanoparticle, and may also be dependent on the shape of the nanoparticle, the size (i.e., diameter) of the nanoparticle, and the thickness of the nanoparticle material of the nanoparticle (e.g., in particular, if the nanoparticle is a nanoshell). However, the ability to calculate the surface plasmon resonance based on these factors is within the skill of the artisan. For example, if the nanoparticle comprises gold, the wavelength of light used to irradiate the nanoparticle will generally be in the range of about 510 nm to about 560 nm, more usually in the 520-530 nm range, and in some embodiments about 532 nm. If the nanoparticle is silver, the light wavelength will generally be in the 420-460 nm range. In contrast, if the nanoparticle comprises a metal oxide, the wavelength of the exciting light will generally be in the near-infrared range.

The detection of a temperature jump associated with a location on the surface of the solid substrate permits the determination of the presence or absence of hybridized DNA. Determining the presence or absence of hybridized DNA can include (i) measuring the generation of heat by the photoinduction of the attached nanoparticle and then (ii) comparing the generated temperature to the temperature by the bare surface.

Detection, measuring and visualizing the thermal event can be detected by any one of a number of photothermal detection methods. The nanoparticles described above provide optical absorption and a photothermal detection system can optionally and further comprise a detector for detecting the optical absorption of the nanoparticles or their environment. In other words, detection of hybridization can be carried out by spectral analysis and thermal analysis.

One apparatus used in spectral analysis is the monochromator. A monochromator is a device for isolating a narrow portion of a spectrum. Several methods are available for measuring thermal changes, including, for example, calorimetry and infrared thermography.

Apparatus which can be used as thermal detectors in thermal analysis include, for example, bolometers, thermopiles, pyroelectrics and micro cantilevers. A bolometer, for example, is a very sensitive thermometer whose electrical resistance varies with temperature and which is used in the detection and measurement of feeble thermal radiation. Bolometers are especially useful in the study of infrared spectra.

Temperature may be measured directly, as with non-contact infrared thermometers, which are commercially available. In some embodiments, a thermocouple attached by a wire to the back side of the solid substrate surface is used to detect the thermal events indicative of nucleic acid hybridization. Alternatively or in addition, various image capture devices known in the art may be used, including fiber coupled photo-diode arrays, photographic film, etc.

In some embodiments, hybridization of nucleic acids is thermally detected by using a charge coupled device. Charge coupled devices (CCD) were initially developed for an electrical analog to magnetic bubble memory. Their analog charge handling capability made them useful in applications other than digital memory storage. Since their development, CCDs have been used to build analog delay lines, transversal filters, Fourier correlators, and signal processors. However, their present primary use has been as solid-state image sensors.

CCDs comprise arrays of thousands of sensor cells that are capable of receiving radiation from multiple wells at the same time. The signals are analyzed, via suitable software, to recover information concerning the target events. As set forth above, CCDs are generally applicable for thermal detection as envisioned by the methods described herein. In general, CCDs consist of closely spaced metal-oxide-semiconductor (MOS) capacitors located on the surface of a semiconductor. With appropriate dopant concentrations and capacitor electrode voltages, a space charge region is formed within the semiconductor directly below the surface of the MOS capacitor. This space charge region generates a potential well that stores charge generated with the material. This charge is generated by a variety of sources, from thermal electrons to injection via the photoelectric effect from photons that are absorbed within the semiconductor. When the voltages of the top electrodes of the MOS capacitors are pulsed in proper sequence, the potential wells move, transporting the stored charge from one MOS capacitor to the next. In this way the CCD becomes an image sensor capable of detecting, storing, and transporting charge generated by incident photons. This powerful concept has not changed over the past decades and the architecture used to implement this concept has changed very little.

CCDs are used in a wide range of image sensing applications, requiring different CCD topologies. The standard, front-illuminated CCD is useful for image sensing in the visible photon energy spectrum and recently in the 1-5 keV x-ray regime. These devices find use in video camera systems and facsimile and image reproduction equipment. Unfortunately this CCD architecture cannot be used in a variety of scientific and industrial applications which require imaging in the blue, ultra-violet, and soft x-ray energy spectrum. This is due to the fact that photons within these spectra are absorbed at the top electrode layer, which is no longer transparent at these energies.

To overcome this problem, thin, back-illuminated CCD was introduced. So-called “back-side-incidence” (BSI) CCD arrays have been proposed for detecting short-wavelength light and certain types of particulate radiation. In these arrays, with respect to each pixel, the light-incidence surface is on a “back” or “rear” surface of the silicon substrate, opposite a “front” surface on which the dielectric layer and the electrodes (gates) of the pixel are formed. In BSI CCD arrays, as noted above, the substrate is usually silicon. The substrate typically is “thin” compared to the thickness of the silicon substrate of a conventional front-side-incidence CCD array (typically 300 μm to 500 μm). The “thin” substrate in a BSI CCD array has a thickness of approximately 10 μm to 20 μm. The substrate is thin in a BSI CCD because short-wavelength light has a relatively large absorption coefficient in silicon. The thin CCD substrate used in a BSI CCD array has low mechanical strength. To increase the strength, a “reinforcing substrate” (made of, e.g., silicon or glass) conventionally is bonded to the “front” surface of the CCD substrate (i.e., the surface on which the electrodes are formed). Such reinforcement prevents damage to the CCD substrate during various subsequent processing steps executed on the surface of the CCD substrate of the BSI CCD array, and facilitates handling during later fabrication processes (such as dicing).

Operationally, BSI CCD arrays must transfer photons efficiently from the light-incidence surface (back surface) of the CCD substrate to the respective depletion regions (charge wells) located on the front surface of the CCD substrate.

In some embodiments, temperature changes caused by target hybridization are detected using infrared thermography. An infrared imaging system, which can be a high resolution infrared imaging system, is used to monitor real time heat output, for example, from a solid surface on which nucleic acid hybridization has been carried out as described herein, with images provided by a central processing unit for data analysis. In some embodiments, an infrared camera is used to monitor the heat generated by hybridization.

Generally, the designation “infrared radiation” refers to electromagnetic radiation having a wavelength of between about 2.5 and about 50 microns or, expressed differently, that having a frequency of between about 200 and about 4000 inverse centimeters (cm⁻¹ or “wave numbers”). The infrared region of the electromagnetic spectrum is sometimes broken down into three sub-regions. These subregions include: (1) the “near-infrared” region spanning wavelengths of about 0.7 to 1.5 microns; (2) the “intermediate-infrared” region including wavelengths of about 1.5 to 20 microns; and (3) the “far-infrared” region covering a range of wavelengths from about 20 to 1000 microns.

Objects above absolute zero (−273° C.) emit electromagnetic radiation in the form of infrared rays. This energy is usually emitted from the first 1/1000 of an inch of the surface of an object. Infrared thermographic instruments are non-contact, non-intrusive systems, that detect only radiated energy from the 1/1000″ of most objects. In general, thermographic instruments, known as either short wave (2-6 micron), or long wave (8-14 micron) systems can see the emitted energy from objects that are at a temperature of approximately −35° C. or higher. Infrared photothermal detection systems are thus not dependent on reflection of visible light or high temperatures.

As understood by those familiar with infrared (IR) radiation and the IR spectrum, the frequencies of electromagnetic radiation generally characterized as infrared are emitted or absorbed by vibrating molecules, and such vibrations generally correspond to the thermal state of a material in relation to its surroundings. Since solid bodies whose temperatures are above absolute zero radiate some infrared energy, and for temperatures up to about 35000K (3227° Celsius, 5840° Fahrenheit), such thermal radiation falls predominantly within the infrared portion of the electromagnetic spectrum. There, thus exists a rather straightforward relationship between the temperature of a body and the infrared radiation which it emits. In some embodiments, the monitoring of radiation in the range of 3-100 microns is preferred, with a range of from about 8 to about 12 microns being generally used. However, other wavelengths or wavelength may also be useful.

The photonic, infrared energy emitted by objects, is focused by a lens onto a specialized detector. The lenses are made of IR-transparent materials, such as germanium, silicon, or zinc selenide, which are generally not transparent to visible light. Detectors in many systems today are un-cooled thermal detectors made from materials such as vanadium oxide, and barium strontium titanate. Other detector materials such as indium antimonide, and indium gallium arsenide are also in use in a variety of infrared thermographic systems.

The light energy that strikes the detector in the camera is usually converted to an electrical impulse. This electrical impulse then produces a video type image on a CRT or LCD display screen in black and white or a series of selectable color pallets. In general, the higher the temperature of the object or area in the field of view, the brighter the color on the screen. Certain cameras can also measure this radiated energy and through the use of an onboard computer give calculated temperatures. This type of thermographic system is called an imaging radiometer or a quantitative infrared system. Infrared thermographic systems that do not calculate temperatures but only display an image are simply called imagers or qualitative devices.

Customarily, the device used for photothermal detection comprises a laser which is focused onto the surface of the part or sample under inspection, in a heating zone. In carrying out IR thermography, a modulated heat source is directed toward the elements on the substrate surface, either directly adjacent to a single element or in such a manner as to simultaneously and uniformly irradiate the entire library. An IR detector scans the array, either by repositioning the detector or by repositioning the substrate surface relative to the detector. The detector monitors the temperature change of array elements in response to the modulation of heat source. If then heat source does not simultaneously and uniformly irradiate the surface, then the surface is generally scanned in conjunction with the detector, thus insuring that the monitored thermal diffusivities correspond to the same heat input.

In some embodiments, the modulated heat source simultaneously and uniformly irradiates the entire substrate surface, and thus all array elements. A position-sensitive IR detector array can monitor the temperature change of all surface or array elements or attachment points. In general, in order to perform a measurement, the sample chamber, solid surface, and IR detection apparatus is first equilibrated to a uniform temperature. At a time t equals 0, the substrate is exposed to light, and the substrate temperature is monitored. Preferably the substrate temperature is monitored at periodic intervals, although continuous monitoring may also be used. The rise or fall in temperature of the thermal mass supporting array elements is a direct measure of the exothermic or endothermic activity of the site.

The radiation source can be directed through individual array elements one-by-one in a serial fashion, or a large area source beam can be passed through the entire library. Similarly, the infrared detection system may be a single infrared detector scanned over the array elements in a serial manner, or it may be a position sensitive imaging system monitoring the absorption of all of the array elements in a parallel manner. In some embodiments, individual elements can be monitored in series using position insensitive temperature detection technology or single element scanned detectors.

Spectral emission light from the target is usually directed through a lens to an optical detector. The optical detector functions, in a manner to be detailed below, to detect one or more emission characteristics of the individual array elements in the illuminated portion of the field. The detector is typically a CCD array which operates to generate and store an array of optical intensity values corresponding to the array pixels. Assuming that the optical system includes a CCD, with a pixel array of photodetecting elements, the width of the central diffraction region, which may cover several pixels, can be measured radially from the peak of the center of the diffraction image to the position in the center of the image where the intensity has fallen to half its peak value (assuming a circular image).

An image processor contained within a computer can be operatively connected to the detector to receive values of light intensity at each of the detector array positions, under each selected illumination condition, e.g., different wavelength. The image processor functions to construct a computer image of the positions and values of one or more spectral emission characteristics measured by the detector. Typically, this is done by treating each pixel in the detector array as a position point in the illuminated field, and assigning to each pixel “position” the light intensity value recorded by that pixel. The image generated by the image processor may be a matrix of stored numbers, e.g., position coordinates and associated spectral emission characteristic value(s), or an actual map in which position are represented, for example, in an x-y plane, and each measured spectral emission value, represented as a quantity along the z axis, for each pixel location.

Direct view systems where the thermal radiation is converted to a visible image (thus eliminating the need for a complex readout system) are also known in the art. Most advanced direct view systems use cantilever bimorph elements which deform in response to thermal radiation. Specifically, a microfabricated bimorph cantilever beam is constructed of two materials that have different coefficients of thermal expansion (CTE). A change in temperature causes the materials to expand or contract by different amounts causing the beam to bend or deform. This deformation can be observed by reflecting visible light, diffracting light from a number cantilevers or by employing any other well-known technique, e.g., from among the ones used in the field of Atomic Force Microscopy. Temperature changes on the order of 10⁻⁵ K. can be detected using bimorph cantilevers for such photothermal spectroscopy. Information about the basic concepts of photothermal spectroscopy using bimorph cantilevers is found in J. K. Gimzewski et al., Chemical Physics Letters, No. 217, 1994, pp. 589.

To provide compatibility with standard television-type raster scan displays, infrared cameras typically include a two-dimensional imaging system for receiving radiant energy. The imaging system usually includes a scanner, which is a device containing a number of reflectors to direct radiant energy from successive elemental areas of an input field of view to one or more radiant energy detectors. Although a single, gimballed reflector might be used to scan the desired field of view, two orthogonal reflectors are generally used, with each reflector being separately driven about a single axis.

Infrared cameras that incorporate scanning mechanisms are known in the art. Scanning mechanisms typically comprise multiple movable reflective surfaces, a drive system to move the reflective surfaces and several lenses to focus incoming IR radiation onto the reflective surfaces. Furthermore, infrared cameras having scanning mechanisms generally cannot support ratiomietric or comparative analysis of target events in each well, since this requires simultaneous image acquisition across all locations on the specimen plate. A focal plane array (“FPA”) is a monolithic microelectronic device that incorporates thousands of sensing elements that continuously receive IR radiation, capturing an image of the entire scene. FPA-based infrared cameras, do not require a scanning system. Rather, they include a single monolithic FPA detector and optics. Consequently, FPA-based cameras are lighter, quieter, consume less power, are more reliable, more, durable and have a lower parts count than scan-based cameras. Furthermore, FPA-based cameras support ratiometric analysis.

The temperature of an entire array of hybridization complexes can thus be monitored with an infrared camera as a measure of the thermodynamic quantities associated with the materials, the measurements performed either serially or in parallel. The speed of the data acquisition from a commercial infrared camera is as high as 120 frames per second, thus providing sufficient speed to follow most chemical reactions and thermal diffusion transients.

High camera sensitivity is required to correctly analyze the signal output. In fact, the continuous component of the signal, representing the scene background, forms a very large part of the signal compared to the variable component which represents the useful information. For example, in conventional 3-5 μm and 8-12 μm infrared spectrum windows, a difference of 1° in temperature between the scene and the background only changes the video signal 1 % compared to the continuous component.

Video cameras employing infrared (IR) sensitive imaging elements can rely on cooled (e.g., cryogenically cooled) IR sensitive focal plane arrays (FPA) or sensors with an associated cryogenic subsystem for maintaining the FPA sensor at temperatures in the range of 60° K. to 80° K. By stabilizing the temperature of the FPA sensor at temperatures which provide substantially zero thermal or infrared radiation emission, any pixel to pixel temperature dependent response variations of individual sensor pixel elements is substantially eliminated since the sensor signal in response to thermal energy generated at cryogenic temperatures is substantially zero. Furthermore, by quickly removing thermal energy absorbed by or conducted to the sensor with the cryogenic subsystem, the signal generated by the sensor can be substantially attributed to the infrared radiation reaching the sensor from a scene to be imaged. Thus a very high signal to noise ratio is maintained.

It is also known to add a hollow cylindrical cold shield over the FPA sensor active surface to define its field of view. Such a cold shield provides a limiting aperture which defines a solid angle field of view of the active surface of the FPA sensor. By also maintained the cold shield at cryogenic temperatures a uniform temperature object having substantially zero infrared radiation emission is provided at the edges of the field of view of each pixel of the FPA sensor active surface. It is also known to provide a cold shield having internal surfaces with high emmissivity for absorbing any stray infrared radiation not incident on the active surface and to provide external surfaces with low emmissivity at infrared wavelengths to reflect any infrared radiation away from the cold shield. Such a cold radiation shield and a method for making is disclosed e.g. by Du Pree et al. in U.S. Pat. No. 5,277,782.

By maintaining the FPA sensor active surface and the cold shield at cryogenic temperatures, a uniform substantially zero DC background signal is provided by each pixel of the FPA sensor active area in the absence of an infrared scene, i.e. when a camera aperture is blocked by a shutter. Cryogenically cooled systems provide good sensitivity to IR radiation at low levels and a high contrast IR image scene signal by providing a high signal to noise ratio.

Alternatively or in addition, microbolometer detector arrays or uncooled IR focal plane arrays (UFPA) operating near room temperature may be used. These systems permit the elimination of cryogenic subsystems An example of an IR video camera employing a UFPA is given by Wood in U.S. Pat. No. 5,420,419. An uncooled infrared sensor assembly comprises an uncooled infrared sensor having an active surface for providing an analog video signal in response to infrared radiation falling thereon. The uncooled infrared sensor assembly can be maintained at a substantially constant temperature in the range of 0° C. to 40° C. and may be an array of microbolometric detector elements or a pyroelectric sensor. Furthermore the TEC may be thermally conductively connected at a first side to the uncooled infrared sensor and at a second opposite side to a first heat sink and heat pipe connected at a first end to the first heat sink draws thermal energy to a second heat sink connected to the heat pipe at a second opposite.

Examples of video electronic signal processing systems are given in U.S. Pat. No. 5,489,776 to Lung and U.S. Pat. No. 5,528,035 to Masarik.

An important component of the IR imaging system is the substantially diffraction-limited optical system, also referred to as the IR microscope. As will be appreciated by those skilled in the art, the optical elements of the microscope have to be substantially transparent at the wavelengths of interest, e.g., 8-12 μm. For the exemplary wavelength range the optical elements typically consist of geranium and/or silicon.

The microscope desirably provides magnification, typically at least 5× (preferably about 10×), and be “fast”, i.e., have a low (50 μm diameter at maximum magnification) object field, in order for the system to be able to image at least a significant portion of a given semiconductor device or assembly.

In some embodiments, a large number of hybridization events can be characterized on an accelerated time scale. The system generally includes a Fourier transform infrared (FT-IR) spectrometer, a high-speed infrared camera, and a computer. In some embodiments configured for operating in a transmission mode, a modified FT-IR spectrometer generates a modulated infrared beam of radiation that is focused onto the array, where it interacts with the hybridization complexes present. After interaction with the attached complexes, the beam is re-focused onto the focal plane array (FPA) of a high-speed infrared camera. The FPA acts as an area detector to capture radiation for every position within the field of view, allowing for true parallel detection of the IR spectra for large arrays, if desired.

In an FT-IR system, light from a point source is rendered parallel by a collimator and passed on to a beamsplitter. The two beams formed by the beamsplitter travel to the mirrors and are reflected back. The beams then recombine at the beamsplitter where they interfere to produce an interferogram that is directed at the array on the surface. After interacting with the surface, the infrared radiation is focused onto the detector. The detector records an intensity signal that depends on the path difference imposed by the travel to and from the mirrors and the absorption by the materials in the array. The distance from the beamsplitter to the mirrors is arbitrary; what is determinative is the difference in the lengths of the paths.

One of the mirror arms in the interferometer is moved at a constant velocity, V. When illuminated by a monochromatic source, the detector will see a periodically varying cosine wave. The electrical frequency f of this wave is determined by the rate of change of the path difference dD/dt. Since dD/dt is simply 2V, f is equivalent to 2 nV. Therefore, a Michelson interferometer can be considered to be a form of frequency transducer that converts optical frequencies which are typically too fast for a detector to monitor down to electrical frequencies that can have any value determined by the mirror velocity V.

The path difference is easily determined with the aid of a laser. The laser beam is sent through the interferometer concurrently with the IR radiation. As the path difference changes, the monochromatic laser light forms a cosine wave at a detector. By counting the number of maxima (fringes) in the pattern generated by the recombined beam, the path difference can be measured very precisely, as is well known in the art.

In some embodiments, the spectroscopic imaging system provides parallel measurement of the infrared spectrum of an entire array of hybridization complexes. Therefore, the modulated IR radiation from the interferometer preferably interacts with each element in the array before it reaches the IR camera.

The FPA of the camera should have a high signal to noise ratio to measure the weak signal coming from each element (i.e., reactor) in the library. The exact limits are set by the amount of intensity provided by the source and by the amount lost in the system. Commercial IR cameras have FPAs made primarily of cooled InSb and HgCdTe detectors with fixed,noise characteristics. Since the camera is typically purchased as a finished package, the sensitivity of the FPA is not the important factor rather, it is the sensitivity of the entire camera (FPA, electronics, filtering, etc.) that is the critical design factor.

In order to track hybridization with the IR apparatus, the IR spectrum can be sampled at certain time intervals that may range from every 5 seconds, or every 20 seconds, or every 30 seconds, or a single measurement depending on the desired information. Capturing the IR spectrum for every element on the substrate surface every several or many seconds requires a high speed IR camera; the data acquisition rate of the camera is determined by the strength of the signal, the desired spectral bandwidth, and the resolution. Although the true time required to obtain a spectrum relies on the data acquisition rate and on the computer processing, the ability of the IR camera to operate at faster than 120 frames/sec allows a sufficient number of interferograms to be sampled to reconstruct the spectrum rapidly.

The images captured by the IR camera can be collected and analyzed to create a series of interferograms (intensity versus time) for each element in the image corresponding to an element on the solid surface. The interferograms must be transformed back to a more useful intensity versus wavelength representation with the aide of a Fourier transform performed by the computer. In order to perform the Fourier transform, the computer must know precisely the time or mirror position corresponding to each image. It is therefore necessary to have an electronic trigger on the interferometer to trigger the camera shutter. In this way a series of plots of absorbance versus wavelength can be constructed for every element within the field of view of the infrared camera.

Several factors are considered in selecting an appropriate IR thermography system. For example, the IR detector must be calibrated to the emissivity of the substrate under observation. Such calibration is easily performed through an independent temperature determination (thermocouple, etc.) prior to the process run. Also, care must be exercised to insure that infrared radiation from another source in the process system, such as a heated electrode, is not transmitted through the sample under observation. Particular apparatus that are useful in the practice of the presently described methods include but are not limited to the infrared imaging systems set forth in U.S. Pat. Nos. 5,483,068 and 6,144,031.

In some embodiments, the hybridization reactions set forth above are carried out in a reaction chamber located within an IR thermography chamber. Following hybridization of a target probe to an array of capture probes on a solid surface, and hybridization of detection probes to any captured target sequences on the surface, the surfaces are thoroughly rinsed in an excess volume of buffer, generally at room temperature. After washing, the surface is prepared for IR thermography detection.

J. Uses And Advantages Of Methods

In a broad aspect, the methods described herein concern a photothermography system for detecting specific target sequences by the use of oligonucleotide probes that are specific for identifying segments of such, acids. These methods have applications in regard to detecting identified nucleic acids in complex mixtures, and are particularly useful for assaying virtually any species so long as an identifiable sequence can be determined. Diagnostic assays, such as for aberrant chromosomal variations, cancers and genetic abnormalities are facilitated by methods described herein to the extent that targeted nucleic acid sequences or segments can be selectively probed employing the described methods.

The described methods can be employed to detect hybridization on an array and can be employed, for example, in sequencing, in mutational analysis (single nucleotide polymorphisms and other variations in a population), and for monitoring gene expression by analysis of the level of expression of messenger RNA extracted from a cell. Thus, examples of the uses of the methods of detecting nucleic acids include the diagnosis and/or monitoring of viral and bacterial diseases, inherited disorders, and cancers where genes are associated with the development of cancer; in forensics; in DNA sequencing; for paternity testing; for cell line authentication; for monitoring gene therapy; and for many other purposes.

Moreover, methods described herein can be employed to monitor hybridization events in a variety of different systems and models. As described more fully below, the present methods are particularly useful in the monitoring of gene expression, the detection of spontaneous or engineered mutations and in the design of probes.

In some embodiments, the present methods can be used to monitor gene expression. In some embodiments, single stranded DNA derived from a gene of interest is used as capture probe. Unexpressed sequences of DNA (for example introns) can be removed before the samples are attached to the support. In this application, it can be desirable to employ cDNA as a probe sequence. Control samples of unrelated single-stranded DNA can also be included to serve as an internal validation of the experiment.

Total mRNA is then isolated from an expression system using standard techniques, which mRNA serves as the target nucleic acid. Target mRNA can optionally be fragmented for ease of handling. The target mRNA is hybridized to the capture probe as described herein. A detection probe comprising a nanoparticle-oligonucleotide complex is then contacted with the support-bound target. In some embodiments of the method, conditions of high stringency are maintained, although these conditions can be varied with the needs and goals of the experiment. The surface can be washed to remove any unhybridized sample.

The surface is then irradiated by a light source, such as a laser. Heat generated by a nanoparticle attached to the surface is measured as set forth above. Gene expression can be determined by comparing duplex formation by the control sequences to duplex formation observed in the target samples. Appropriate mathematical descriptions and treatments of the observed duplex formation can indicate the degree of observed hybridization and consequently the degree of gene expression.

In some embodiments, the present methods can also be employed in the detection of mutations in a nucleic acid sequence. Such mutations can engineered or spontaneous. For example, the present methods can be useful in determining whether an engineered mutation is present in a nucleic acid sequence, or for determining if a nucleic acid sequence contains deviations from its wild type sequence.

In these embodiments, single-stranded oligonucleotide probes are initially prepared. The probes can be known or suspected to contain a mutation(s) to be identified. Capture probe samples are attached to the support using methods described herein. Nucleic acid target sequences to be screened for the mutation are isolated from an expression system, and single stranded target sequences are prepared. If desired, large quantities of sample can be conveniently prepared using established amplification methods, as set forth above. Probe sequences are bound to a nanoparticle to form a detection probe, which is contacted with the capture probe-target hybridization complexes. Those probe sequences containing the mutation of interest will hybridize with the target sequence to form detectable complexes. Unbound target sequences can be removed by washing. The support, which can comprise any formed duplexes, is then irradiated by light and the resulting thermal events detected. In some embodiments, a mutation can be located on either a target sequence or on a probe sequence, the selection of which can be made during experimental design.

In some embodiments, the present methods can be employed in designing nucleic acid probes. The ability to detect hybridization events permits a researcher to optimize a probe for the needs of a given experiment. For example, a probe can be designed that will accommodate a degree of polymorphism in a target sample. Such a probe can be useful for screening for genes or sequences known to exhibit polymorphisms. Using the present invention, it is possible to design a probe that will tolerate a degree of uncomplementarity in the sequence.

Additionally, the present methods can be used to screen for duplex formation between a target sequence and a polymorphic probe; that is, a probe that has one or more mutations from the wild type sequence. By varying the number of bases different from the wild type sequence, a desired degree of promiscuity in a probe can be obtained.

In this context, the present methods can be useful for detecting hybrid formation in sequential rounds of probe design. For example, if a designed probe binds only to the wild type sequence, no polymorphism is recognized; if the probe binds to sequences unrelated to the target sequence, the probe is not useful to identify the sequence of interest. By monitoring hybrid formation at each round of optimization, the present invention can be useful for nucleic acid probe design.

Photothermography detection methods of the present invention offer significant advantages over detection systems known in the art. One particular advantage is the elimination of any requirement for individually wired sample cells. Commercially available microarray supports suitable for electrochemical detection of nucleic acid duplexes require that each sample be attached to the support at a different electrode. That is, duplex formation at each attachment point must be monitored by detecting a current through an electrode dedicated to each individual cell. The present invention can employ only a single electrode and achieves detection at each capture probe attachment point on the electrode by detecting and optionally imaging thermal variation across the entire surface by a light source.

### EXAMPLES

The following Examples have been included to illustrate some modes of the invention. Certain aspects of the following Examples are described in terms of techniques and procedures found or contemplated by the present inventors to work well in the practice of the invention. In light of the present disclosure and the general level of skill in the art, those of skill will appreciate that the following Examples are intended to be exemplary only and that numerous changes, modifications and alterations can be employed without departing from the spirit and scope of the invention.

### Example 1

### X-Ray Photoelectron Spectroscopy Characterization of ITO Electrode Surfaces Modified By Single Stranded DNA And Gold Nanoparticles

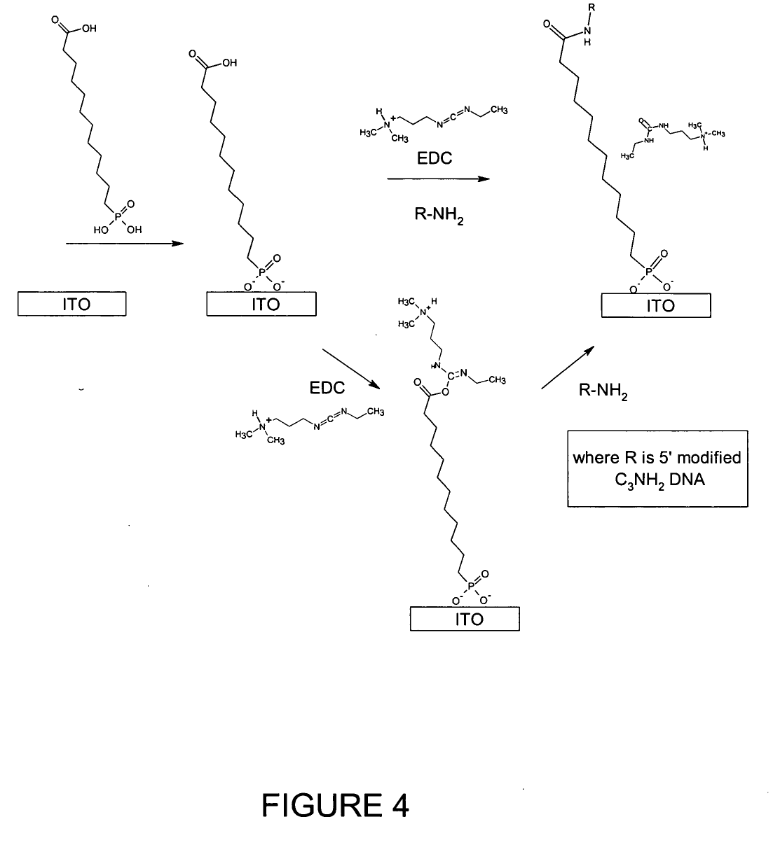

FIG. 4 outlines one strategy employed in the modification of indium tin oxide (ITO) with single-stranded DNA (ssDNA). Initially, a monolayer of 12-phosphonododecanoic acid (10 mM in 50/50 DMSO/18 MΩ cm H₂O for 16 hours) was formed on the ITO surface (cleaned 20 minutes with UV/O₃ (UVO-cleaner (UVO-60), model number 42, Jelight Company, Inc.)). The carboxylic acid of 12-phosphonododecanoic acid was then activated by 1-ethyl-3-(3-dimethylaminopropyl)carbodiimide hydrochloride (EDC) to form an O-acylisourea intermediate. See, e.g., S. H. Brewer et al., Langmuir (2002) 18, 6857-6865; B. L. Frey and R. M. Corn, Analytical Chemistry (1996) 68, 3187-3193; M. Burgener et al., Bioconjugate Chemistry (2000) 11, 749-754; K. Kerman et al., Analytica Chimica Acta (2002) 462, 39-47; E. Huang et al., Langmuir (2000) 16, 3272-3280; and G. T. Hermanson, Bioconjugate Techniques (1996) (Academic Press: San Diego).

This activated carboxylic acid group is attacked by the primary amine (acting as a nucleophile) of a 5′-modified C₃NH₂ ssDNA strand to form an amide bond between the monolayer of 12-phosphonododecanoic acid and the 5′ modified C₃NH₂ ssDNA. The coupling conditions were 1 μM 5′-modified C₃NH₂ ssDNA and 200 mM EDC for 4 hours in a 0.1 M MES (2-(N-morpholino)ethane sulfonic acid) buffer at pH 5 with 0.25M NaCl.

Complementary 18-base pair single-stranded DNA sequences were attached to ITO and 10 nm diameter gold nanoparticles according to the foregoing methods.

X-ray photoelectron spectroscopy (XPS) spectra were recorded on a Riber LAS 2000 Surface Analysis System equipped with a cylindrical mirror analyzer (CMA) and a MAC2 analyzer with Mg Kα X-rays (model CX 700 (Riber source) (hν=1253.6 eV). The elemental scans had a resolution of 1.0 eV and were the result of 5 scans. XPS spectra were smoothed using a 9 point (second order) Savitzky-Golay algorithm, baseline corrected and the peaks were fitted using Gaussian line shapes.

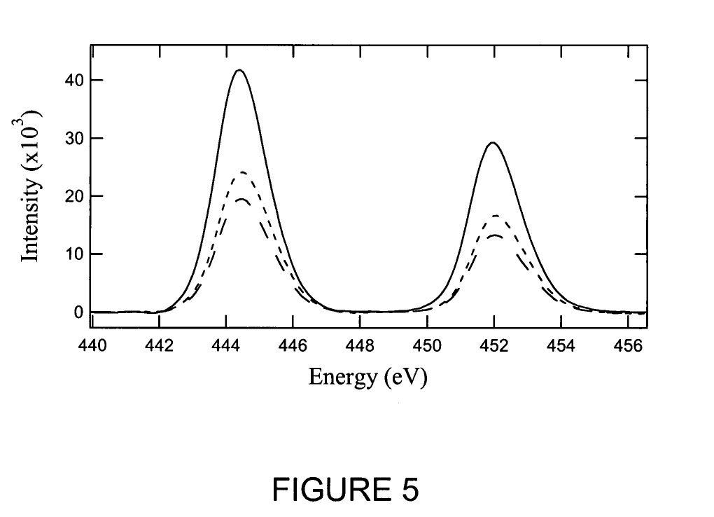

The results of these experiments is shown in FIGS. 5, 6, 7 and 8.

FIG. 5 is the x-ray photoelectron spectra (XPS) of In 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

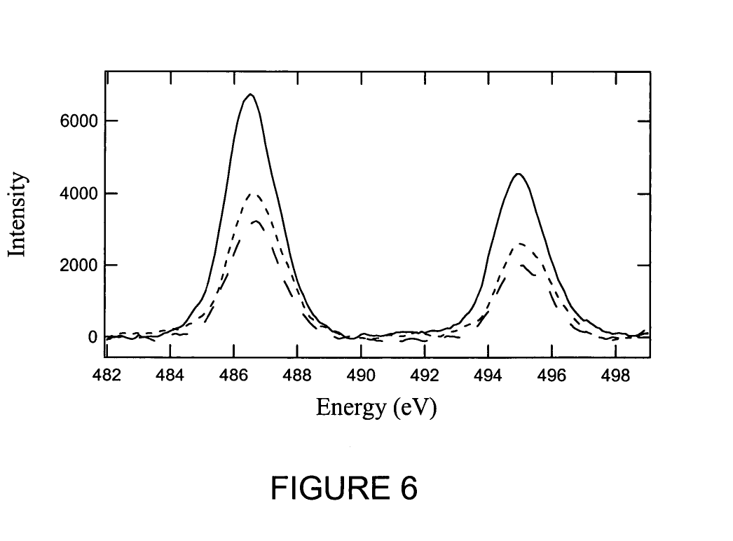

FIG. 6 is the XPS spectra of Sn 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

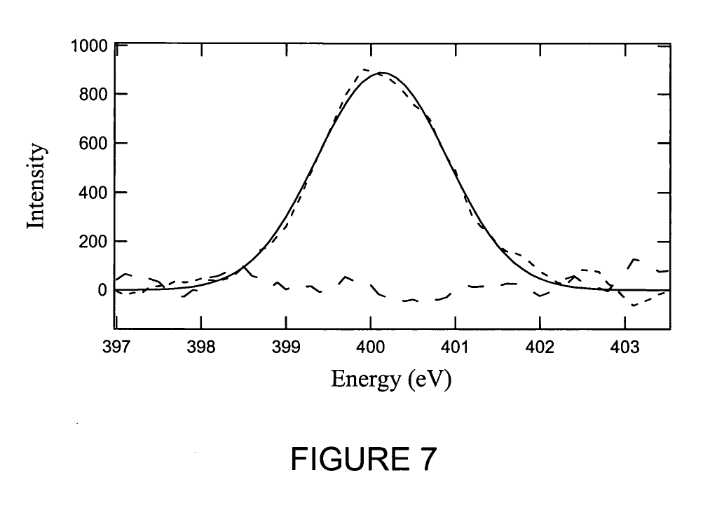

FIG. 7 is the XPS N 1s spectra of ITO modified with a monolayer of 12-phosphonododecanoic acid (long dash) and ITO modified with ssDNA coupled through a monolayer of 12-phosphonododecanoic acid (short dash) fitted to a Gaussian line shape (solid).

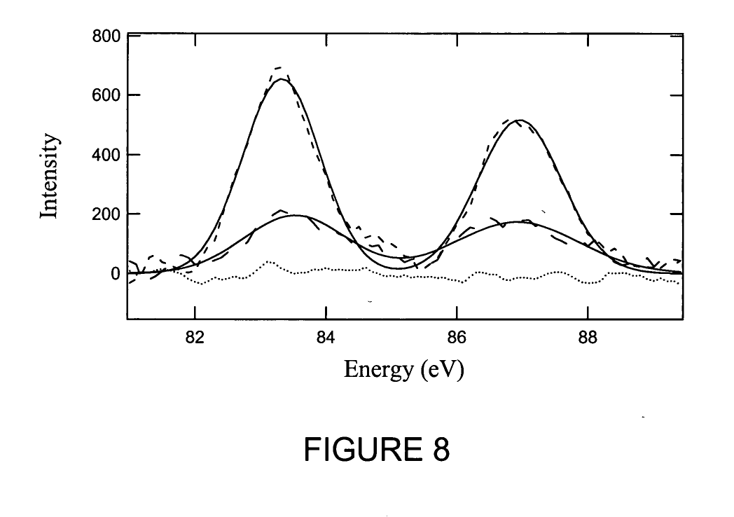

FIG. 8 is the XPS Au 4f_{7/2,5/2} spectra of ITO modified with ssDNA coupled through a monolayer of 12-phosphonododecanoic acid (dotted line) exposed to the complementary (short dash) or non-complementary (long dash) ssDNA labeled with a 10 nm gold nanoparticle (1 nM) fitted to two Gaussian line shapes (solid).

### Example 2

### Infrared Reflection Absorption Spectroscopy (IRRAS)

The reflectance FTIR spectra were recorded using a Spectra-Tech grazing angle reflectance attachment in a Nicolet Magna-IR 860 FTIR spectrometer. The angle of incidence used was 80 degrees. An infrared polarizer was used to p-(vertically) polarized light. The spectra of the monolayers deposited on the ITO surfaces were obtained by taking a ratio of the single beam spectra of the deposited material on an ITO surface to one of a clean ITO surface. The rotational lines from gaseous water were subtracted from these spectra. The FTIR spectrometer was equipped with a liquid nitrogen cooled MCT/A detector and the spectra were recorded at a resolution of 2 cm⁻¹ with a spectral range of 900-4000 cm⁻¹. All IR spectra were the result of 256 scans and were recorded at room temperature.

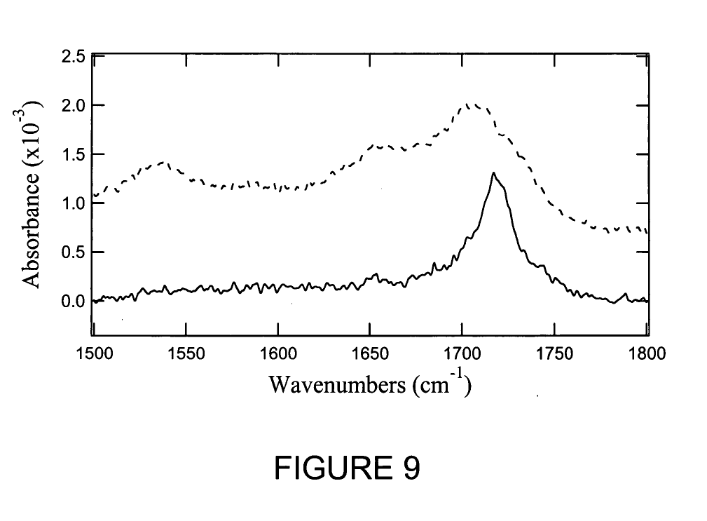

The results of these experiments are illustrated in FIG. 9. FIG. 9 shows a grazing angle reflectance FTIR spectra of ITO modified with a monolayer of 12-phosphonododecanoic acid (solid) coupled to ssDNA (dashed) recorded at an incident angle of 80 degrees with p-polarized radiation.

### Example 3

### LITJ At Gold Nanoparticle-Coated ITO Electrodes

LITJ was demonstrated by attaching 10 nm diameter gold particles to ITO via aminosilane linkers, according to the method of K. C. Grabar et al., J. Am. Chem. Soc. (1996) 118, 1148. Visible spectroscopy revealed a particle coverage of 1.5×10¹⁰/cm². Temperature was then monitored during illumination with 532 nm light from a frequency doubled YAG laser (Coherent Antares 76-YAG laser).

The temperature change at the electrode surface was measured using an internal electrochemical standard consisting of 100 mM ferrocene and 0.1 mM ferrocinium in acetonitrile (Aldrich). The temperature dependence of this redox couple was first measured with a 2-compartment electrochemical cell and hot plate to be 0.35 mV °C⁻¹. When the solution was placed in contact with a gold nanoparticle-coated ITO electrode and irradiated at 532 nm, a 9 mV change was recorded. This value corresponds to an interfacial ΔT of 25° C. induced by the LITJ effect.

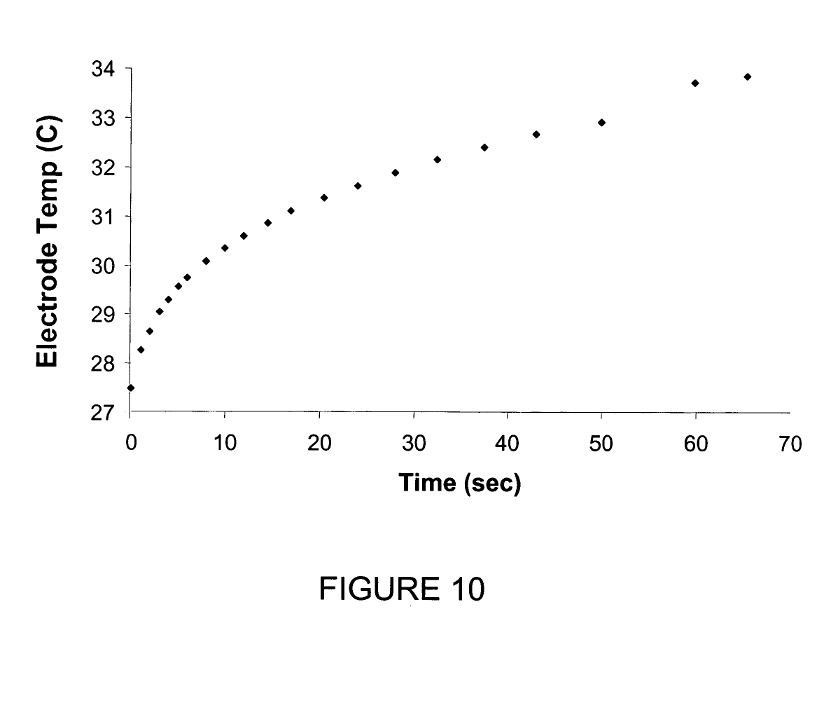

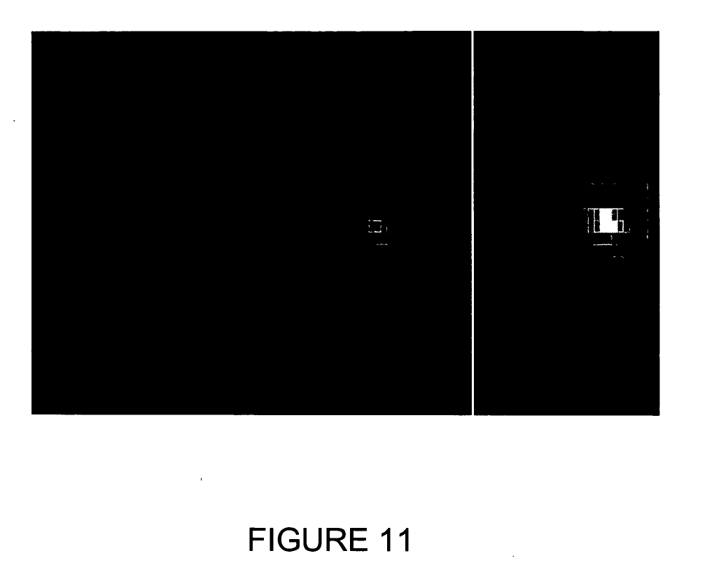

The temperature change at the electrode surface was confirmed by infrared thermography (Inframetrics 740). Following 30 seconds of irradiation, a surface temperature of 42.9° C. was measured for a particle coverage of 3.5×10¹⁰ particles cm⁻². FIG. 10 is a graph showing the increase in electrode temperature as a function of time. FIG. 11 shows a series of infrared thermograms (8 μm -12 μm) of gold nanoparticle-coated glass slides under irradiation with 532 nm light (16 W/cm²). Particle densities were 1×10¹⁰ cm⁻², 2×10¹⁰ cm⁻², and 3.5×10¹⁰ cm⁻² for A, B, and C, with recorded temperatures of 30.5° C., 35.3° C., and 42.9° C., respectively. Light-off temperature was 24.6° C. (ΔT for bare glass was <2° C.). In other experiments, temperature changes of 2.5° C. for as few as 10,000 nanoparticles (10⁶ cm⁻²) have been recorded using IR thermography, as described below.

### Example 4

### Infrared Thermography Of Gold Nanoparticles

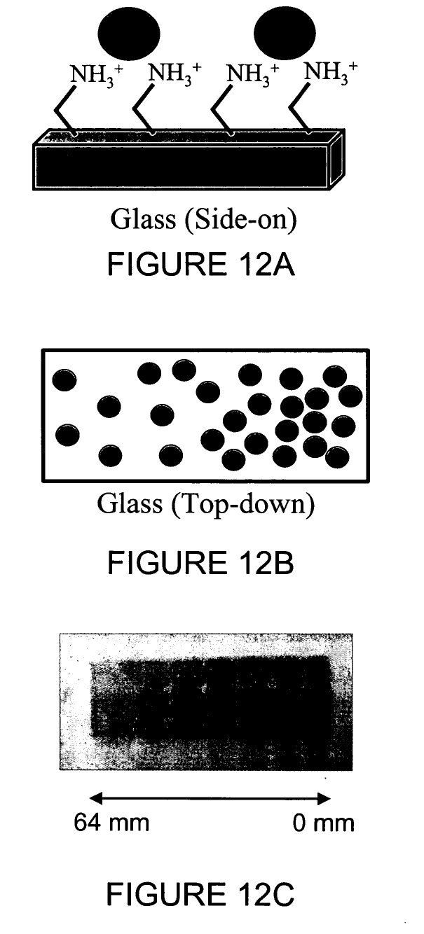

FIG. 12A is a schematic drawing illustrating a side view of gold nanoparticles attached to the surface of a glass slide using an aminosilane linkage.

FIG. 12B is a schematic drawing of an overhead view of the glass slide of FIG. 12A, in which gold nanoparticles have been attached in a graded density pattern to a glass slide. Particle density is greatest at the right-most end of the slight, becoming decreasingly dense towards the left-most end.

FIG. 12C is a digital photograph of the glass slide illustrated schematically in FIGS. 12A and 12B. As indicated, the slide is 64 mm in length, and density of the attached nanoparticles is greatest at the “0 mm” end, and least dense at the “64 mm” end.

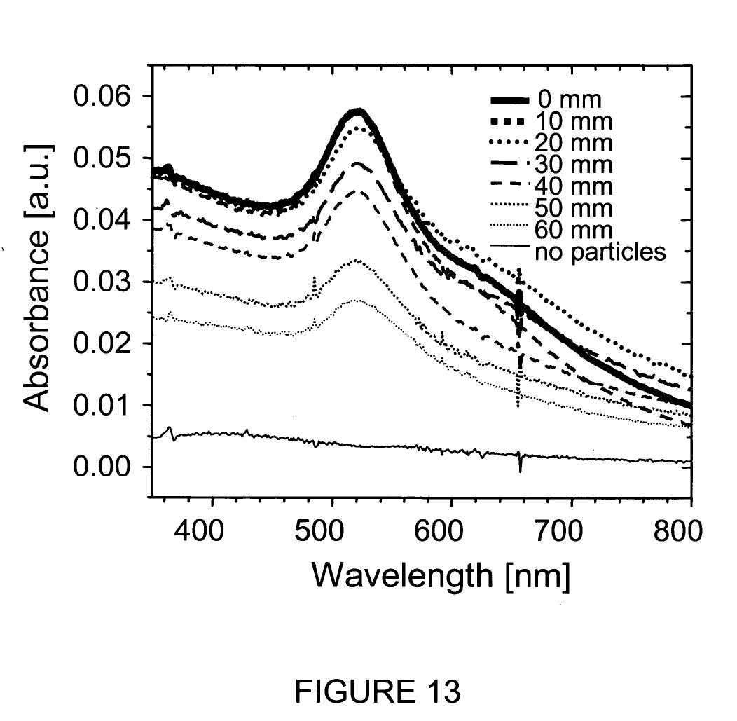

FIG. 13 is a graph of light absorbance as a function of light wavelength for the attached gold nanoparticles described in FIG. 12C. That is, nanoparticles towards the end of the slide with the greatest density (“0 mm”) exhibited the overall higher absorbance, while nanoparticles towards the end of the slide with the least density (“64 mm”) exhibited the lowest, with the sample containing no particles having the lowest absorbance, as expected. All samples exhibit a marked and detectable absorbance peak at around 532 nm, where the light wavelength matches the surface plasmon resonance of the gold nanoparticles.

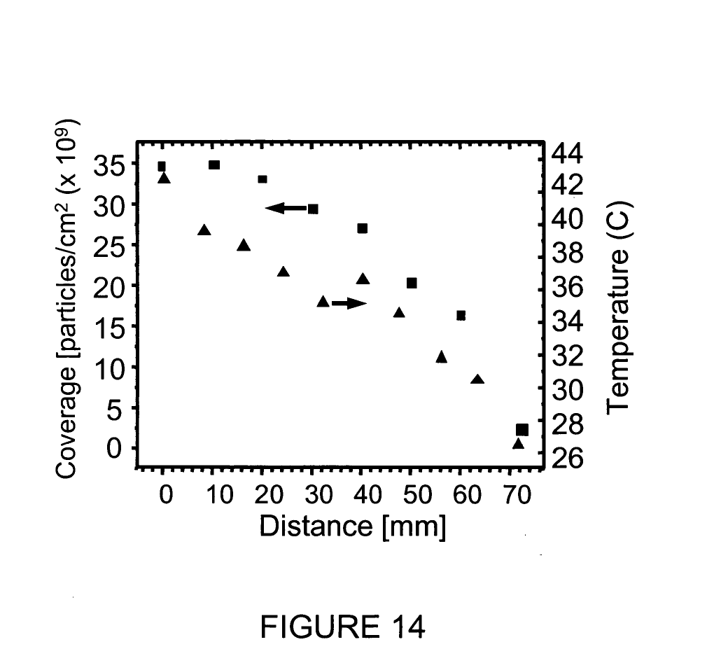

FIG. 14 is a graph of gold nanoparticle density (square data points) and temperature detected after irradiation by a laser at 532 nm (triangular data points), as a function of physical slide location as described in FIG. 12C. Consistent with the results shown in FIG. 13, nanoparticles towards the end of the slide with the greatest density (“0 mm”) were present in the greatest density and exhibited the highest detected temperatures, which while nanoparticles towards the “64 mm” end of the slide were present in the lowest particle density and exhibited the lowest temperatures. The temperature jump (Δ T) measured for nanoparticle sample comprising 3.1×10⁵ nanoparticles was 2.5° C.

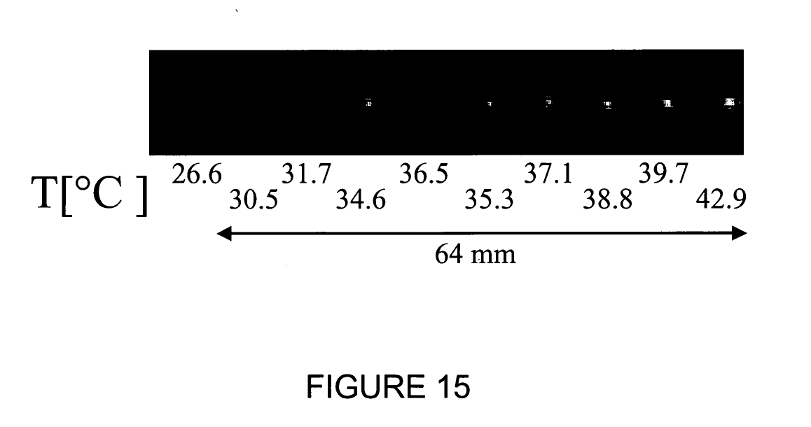

FIG. 15 is an infrared thermogram of the slide described in FIGS. 12, 13, and 14. Taken together, these four figures indicate that the amount of heat generated by gold nanoparticles attached to a solid surface and irradiated at a wavelength equal to their surface plasmon resonance is directly and predictably proportional to the concentration of nanoparticles attached at the surface.

### Example 5

### DNA Detection With Infrared Thermography

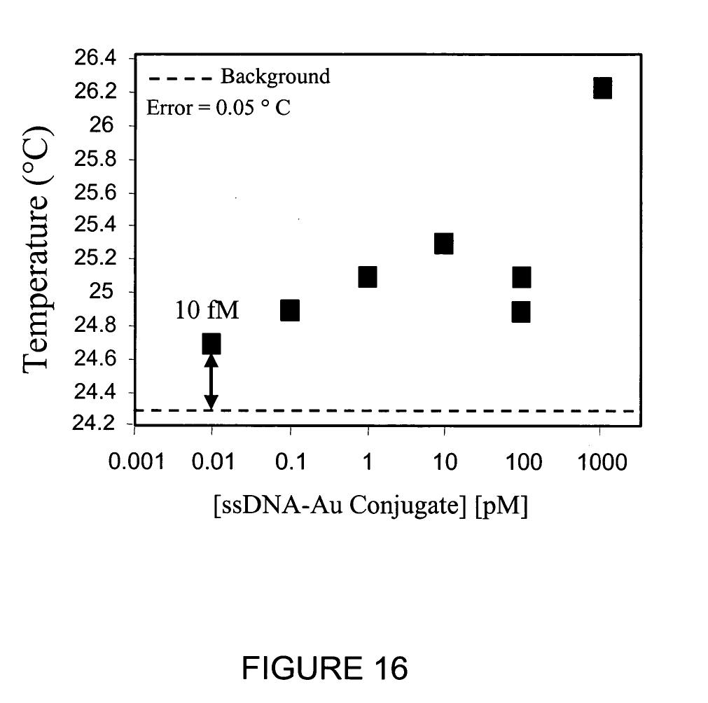

FIG. 16 is an illustration of the limits of detection for single stranded DNA conjugated to gold nanoparticles for infrared thermography detection as described herein. In FIG. 16, temperature is graphed as a function of ssDNA-nanoparticle conjugate concentration in pM, while the background temperature of the solid substrate is indicated as a broken line. These results indicate that the limit of detection for this method is on the order of 10 fM.

It will be understood that various details of the claimed subject matter can be changed without departing from the scope of the claimed subject matter. Furthermore, the foregoing description is for the purpose of illustration only, and not for the purpose of limitation.

What is claimed is:

1. A method of detecting a target nucleic acid sequence, comprising:
providing a hybridization complex comprising (a) a capture probe that is attached to a solid surface and (b) a target nucleic acid sequence that is hybridized to the capture probe, wherein the target nucleic acid sequence additionally comprises at least one nanoparticle attached to the target nucleic acid sequence;
exposing the solid surface to light at a wavelength absorbed by the nanoparticle; and
detecting a temperature of the solid surface, whereby detection of an increased temperature relative to a temperature of the solid surface that would be detected in the absence of said complex indicates the presence or amount of target nucleic acid sequence hybridized to the solid surface.

2. The method according to claim 1, comprising:
hybridizing a target sequence to at least one capture probe to form a first hybridization complex, wherein the capture probe is attached to a solid surface;
hybridizing a detection probe to the first hybridization complex to form a second hybridization complex, wherein the detection probe comprises a nanoparticle;
exposing the solid surface to light at a wavelength absorbed by the nanoparticle; and
detecting the temperature of the solid surface at the attachment location of the capture probe, wherein an increase in temperature at the attachment location as compared to the background temperature of the solid surface indicates hybridization of the target sequence to the solid surface.

3. The method of claim 1, wherein the target sequence comprises RNA.

4. The method of claim 1, wherein the target sequence comprises cDNA.

5. The method according to claim 1, wherein the solid surface comprises indium tin oxide.

6. The method according to claim 1, wherein the target sequence is present in a biological sample.

7. The method according to claim 2, wherein the detection probe comprises a nanoparticle comprising one or more of metals and metal oxides.

8. The method according to claim 7, wherein the nanoparticle comprises a metal comprising one or more of gold, silver, and platinum.

9. The method according to claim 1, wherein the nanoparticle comprises gold.

10. The method according to claim 1, wherein the nanoparticle is a nanoshell.

11. The method according to claim 1, wherein the nanoparticle has a diameter from about 10 to about 20 nanometers.

12. The method according to claim 1, wherein the nanoparticle exhibits surface plasmon resonance, and wherein the solid surface is exposed to light at a wavelength that matches the surface plasmon resonance of the nanoparticle.

13. The method according to claim 1, wherein the light is generated by a laser.

14. The method according to claim 2, wherein the detection probe further comprises an oligonucleotide attached to the nanoparticle.

15. The method according to claim 14, wherein the capture probe is complementary to a first target domain of the target sequence, and the detection probe oligonucleotide is complementary to a second target domain of the target sequence.

16. The method according to claim 2, wherein the detection probe comprises a nanoparticle attached to one partner of a ligand-binding pair, and the target sequence comprises the other partner of a ligand-binding pair.

17. The method according to claim 16, wherein one partner of a ligand-binding pair is streptavidin, and the other partner of the ligand-binding pair is biotin.

18. The method according to claim 16, wherein the target sequence comprises biotin.

19. The method according to claim 18, wherein the biotin has been incorporated into the target sequence during nucleic acid amplification.

20. The method according to claim 18, wherein the detection probe comprises a nanoparticle attached to streptavidin.

21. The method according to claim 1, wherein a plurality of different capture probes are attached to the solid surface in an array, and the location of each capture probe comprises an array element.

22. The method according to claim 21, wherein each array element is exposed to light separately.

23. The method according to claim 21, wherein the entire plurality of capture probes is exposed to light simultaneously.

24. The method according to claim 1, wherein the light is provided by a light source is selected from the group consisting of a tungsten halogen light source, a xenon arc lamp and a laser.

25. The method according to claim 1, where in the exposing is by rastering.

26. The method according to claim 1, wherein the target sequence is selected from the group consisting of an mRNA sequence derived from a sample and a cDNA sequence derived from a sample.

27. The method according to claim 1, wherein the capture probe comprises a sequence from a gene of interest.

28. The method according to claim 1, wherein the capture probe comprises or is suspected to comprise a mutation to be detected

29. The method according to claim 1, wherein the target sequence comprises or is suspected to comprise a mutation to be detected

30. The method according to claim 1, wherein the nanoparticle comprises silver and the solid surface is exposed to light at a wavelength ranging from about 420-460 nm.

31. The method according to claim 1, wherein the nanoparticle comprises gold and the solid surface is exposed to light at a wavelength of about 532 nm.

32. The method according to claim 1, wherein the detecting step is carried out by a thermocouple attached to a side of the solid surface upon which capture probes are not attached.

33. The method according to claim 1, wherein the detecting step is carried out by infrared thermography.

34. The method according to claim 1, wherein the detecting step is carried out by Fourier Transform infrared thermography.

35. The method according to claim 1, wherein the detecting step comprises capturing a thermal image by means of an infrared camera.

36. The method according to claim 1, wherein the detecting step is carried out by a charge coupled device.

37. The method according to claim 1, wherein the nanoparticle is attached to the target sequence.

38. The method according to claim 37, where the nanoparticle is attached to the target sequence by one of a binding pair and complementary nucleic acids.

39. The method according to claim 37, where the nanoparticle is attached to the target sequence by one of primer extension and ligation of a nanoparticle-labeled nucleic acid.

40. The method of claim 1, wherein the complex comprises a detection probe.

41. The method of claim 40, wherein the detection probe is attached to the target sequence before, during, or after the target sequence hybridizes to the capture probe.

42. The method of claim 40, comprising the sequential steps of hybridizing the target to the capture probe; and then reacting the hybrid with a detection probe.
